(19) United States
(12) Patent Application Publication
(10) Pub. No.: US 2024/0358711 A1
(43) Pub. Date: Oct. 31, 2024

(54) COMPOUNDS FOR NEUTROPHIL ROS INHIBITION

(71) Applicants: IMMUNYX PHARMA LTD., Jerusalem (IL); YISSUM RESEARCH DEVELOPMENT COMPANY OF THE HEBREW UNIVERSITY OF JERUSALEM LTD., Jerusalem (IL); Hadasit Medical Research Services and Development Ltd., Jerusalem (IL)

(72) Inventors: Seth Jonah SALPETER, Jerusalem (IL); Meital NAIM, Jerusalem (IL); Zvika GRANOT, Jerusalem (IL); Zvi Gregorio FRIDLENDER, Zur-Hadassah (IL)

(73) Assignees: IMMUNYX PHARMA LTD., Jerusalem (IL); YISSUM RESEARCH DEVELOPMENT COMPANY OF THE HEBREW UNIVERSITY OF JERUSALEM LTD., Jerusalem (IL); Hadasit Medical Research Services and Development Ltd., Jerusalem (IL)

(21) Appl. No.: 18/292,918

(22) PCT Filed: Jul. 28, 2022
(86) PCT No.: PCT/IL2022/050823

§ 371 Date: Jan. 28, 2024

Related U.S. Application Data
(60) Provisional application No. 63/226,297, filed on Jul. 28, 2021.

(51) Int. Cl. A61K 31/55; A61K 31/137; A61K 31/4706; A61K 31/506; A61K 31/517; A61K 31/519; A61K 31/5415; A61K 45/06; A61K 47/64; A61P 29/00

(52) U.S. Cl. CPC A61K 31/55; A61K 31/137; A61K 31/4706; A61K 31/506; A61K 31/517; A61K 31/519; A61K 31/5415; A61K 45/06; A61K 47/64; A61P 29/00

(57) ABSTRACT

Methods of inhibiting reactive oxygen species production by neutrophils or treating neutrophil-mediated inflammation in a subject by contacting the neutrophils with or administering an agent selected from a cyclin dependent kinase 4 (CDK4) inhibitor, a cyclin dependent kinase 6 (CDK6) inhibitor, an epidermal growth factor receptor (EGFR) inhibitor, a proto-oncogene tyrosine-protein kinase Src (SRC) inhibitor and a sodium (Na) channel blocker are provided. Pharmaceutical compositions comprising a nanoparticle, the agent and a neutrophil targeting peptide are also provided.

### CROSS-REFERENCE TO RELATED APPLICATIONS

This application claims the benefit of priority of U.S. Provisional Patent Application No. 63/226,297 filed Jul. 28, 2021, the contents of which are incorporated herein by reference in their entirety.

### FIELD OF INVENTION

The present invention is in the field of neutrophil pathology.

### BACKGROUND OF THE INVENTION

Neutrophils are potent effector cells in a wide range of infectious, inflammatory and autoimmune conditions as well as cancer. In certain instances, the neutrophils' natural role as cytotoxic, inflammation causing cells can have adverse consequences. Diseases characterized by excessive neutrophil-mediated tissue damage require therapeutics that can temporarily inhibit the neutrophils' effector functions, but without causing neutrophil cell death and neutropenia. Therapeutics that can specifically and strongly inhibit neutrophil effector function while not killing neutrophils are greatly needed.

### SUMMARY OF THE INVENTION

The present invention provides methods of inhibiting reactive oxygen species (ROS) production by neutrophils or treating neutrophil-mediated inflammation in a subject by contacting the neutrophils or administering an agent selected from a cyclin dependent kinase 4 (CDK4) inhibitor, a cyclin dependent kinase 6 (CDK6) inhibitor, an epidermal growth factor receptor (EGFR) inhibitor, a proto-oncogene tyrosine-protein kinase Src (SRC) inhibitor and a sodium (Na) channel blocker. Pharmaceutical compositions comprising a nanoparticle, the agent and a neutrophil targeting peptide are also provided.

In one aspect of the invention, there is provided a method of inhibiting reactive oxygen species (ROS) production by neutrophils, the method comprising inhibiting cyclin dependent kinase 4 (CDK4), cyclin dependent kinase 6 (CDK6), epidermal growth factor receptor (EGFR), proto-oncogene tyrosine-protein kinase Src (SRC) or a sodium (Na) channel in said neutrophils, thereby inhibiting ROS production.

In one embodiment, the method comprising contacting said neutrophils with an agent selected from: a CDK4 inhibitor, a CDK6 inhibitor, an EGFR inhibitor, a SRC inhibitor and a Na channel blocker.

In another aspect, there is provided a method of treating neutrophil-mediated inflammation in a subject in need thereof, the method comprising administering to said subject an agent selected from: a CDK4 inhibitor, a CDK6 inhibitor, an EGFR inhibitor, a SRC inhibitor and a Na channel blocker; thereby treating neutrophil-mediated inflammation in a subject.

In one embodiment, neutrophil-mediated inflammation comprises neutrophil-mediated tissue damage.

In one embodiment, the inflammation, tissue damage or both is mediated by ROS production by neutrophils.

In one embodiment, the inflammation, tissue damage or both comprises elevated ROS from neutrophils.

In one embodiment, the subject suffers from a disease or condition selected from an inflammatory disease, an autoimmune disease, cancer, acute tissue damage and an infectious disease.

In one embodiment, the disease or condition is selected from acute respiratory distress syndrome (ARDS), chronic obstructive pulmonary disease (COPD), acute lung injury (ALI), inflammatory bowel disease (IBD), sepsis, bacterial pneumonia, and viral pneumonia.

In one embodiment, the subject suffers from lung inflammation.

In one embodiment, the subject suffers from ARDS or COPD.

In one embodiment, the agent is a CDK4/6 dual inhibitor.

In one embodiment, the CDK4/6 dual inhibitor is selected from Abemaciclib, Ribociclib and Palbociclib.

In one embodiment, the agent is an EGFR inhibitor.

In one embodiment, the EGFR inhibitor is selected from Afatinib and Trifluoperazine.

In one embodiment, the agent is a SRC inhibitor.

In one embodiment, the SRC inhibitor is Bosutinib.

In one embodiment, the agent is a Na channel blocker.

In one embodiment, the Na channel blocker is selected from Dyclonine, Trifluoperazine and a tricyclic antidepressant (TCA).

In one embodiment, the TCA is selected from Clomipramine, and Amitriptyline.

In one embodiment, the method further comprises inhibiting elastase secretion from said neutrophils, optionally by administering an elastase secretion inhibitor.

In one embodiment, the agent is linked to or encapsulated by a nanoparticle.

In one embodiment, the nanoparticle comprises a neutrophil targeting moiety.

In one embodiment, the targeting moiety comprises a peptide selected from SEQ ID NO: 1-13.

In one embodiment, the neutrophils are human neutrophils and said peptide is selected from SEQ ID NO: 1-8.

In one embodiment, the neutrophils are rodent neutrophils and said peptide is selected from SEQ ID NO: 9-13.

In one embodiment, inhibiting is inhibiting ROS production by activated neutrophils.

In another aspect, there is provided a pharmaceutical composition comprising a nanoparticle comprising an agent and a neutrophil targeting peptide; wherein said agent is selected from a cyclin dependent kinase 4 (CDK4) inhibitor, a cyclin dependent kinase 6 (CDK6) inhibitor, an epidermal growth factor receptor (EGFR) inhibitor, a proto-oncogene tyrosine-protein kinase Src (SRC) inhibitor and a sodium (Na) channel blocker; and wherein said neutrophil targeting peptide comprises a sequence selected from SEQ ID NO: 1-13.

In one embodiment, the agent is selected from Afatinib, Clomipramine, Nisoldiprine, Amitriptyline, Trifluoperazine, Abemaciclib, Ribociclib, Palbociclib Bosutinib, and Dyclonine.

In one embodiment, the agent is Trifluoperazine.

In another aspect, there is provided a method for treating neutrophil-mediated inflammation in a subject in need thereof, the method comprising administering to said subject a compound represented by Formula 1:

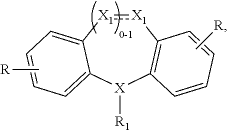

including any salt thereof; wherein each R is independently H, or represents one or more substituents independently comprising any one of —NO₂, —CN, —OR′, —CONH₂, —CONR′₂, —CNNR′₂, —CSNR′₂, —CONH—OR′, —CONH—NR′₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NR′NR′₂, —NR′NR′—OR′, —NNR′, carbonyl, C₁-C₁₀ haloalkyl, optionally substituted C₁-C₁₀ alkyl, —NH₂, —NR′₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted, as allowed by valency; each X and X1 is independently selected from C, CR1, CH, CH2, N, NR1, NH, O, and S; each R1 is independently H, or

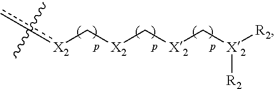

wherein each X₂ and X′₂ is independently selected from CR, CH, CH₂, N, NR, NH, O, and S, as allowed by valency, or is absent; optionally wherein any one of (i) two X2, (ii) two X′2 are interconnected by a covalent bond, forming a carbocyclic ring, optionally comprising one or more heteroatoms; p is between 0 and 10; each R2 is independently H, absent, or is selected from the group comprising an optionally substituted C1-C10 alkyl, an C1-C10 alkyl-aryl, an C1-C10 alkyl-cycloalkyl, optionally substituted C3-C10 cycloalkyl, optionally substituted C3-C10 heterocyclyl, optionally substituted heteroaryl, optionally substituted aryl, —NO2, —CN, —OR′, —CONH2, —CONR′2, —CNNR′2, —CSNR′2, —CONH—OR′, —CONH—NR′2, —NHCOR′, —NHCSR′, —NHCNR′, —NC(═O)R′, —NC(═O)OR′, —NC(═O)NR′, —NC(═S)OR′, —NC(═S)NR′, —SO2R′, —SOR′, —SR′, —SO2OR′, —SO2N(R′)2, —NR′NR′2, —NR′NR′—OR′, —NNR′, carbonyl, C1-C10 haloalkyl, optionally substituted C1-C10 alkyl, —NH2, —NR′2, —NH(C1-C10 alkyl), —N(C1-C10 alkyl)2, C1-C10 haloalkoxy, hydroxy(C1-C10 alkyl), hydroxy(C1-C10 alkoxy), alkoxy(C1-C10 alkyl), alkoxy(C1-C10 alkoxy), amino(C1-C10 alkyl), —CONH(C1-C10 alkyl), —CON(C1-C10 alkyl)2, —CO2H, —CO2R′, —OCOR′, —C(═O)R′, —OC(═O)OR′, —OC(═O)NR′, —OC(═S)OR′, —OC(═S)NR′, a heteroatom, (C1-C10 alkyl)alkyl-cycloalkyl, (C1-C10 alkyl)alkyl-aryl, (C1-C10 alkyl)alkyl-heteroaryl, or a combination thereof as allowed by valency; or wherein both R2 are interconnected by a covalent bond, forming a carbocyclic ring, optionally comprising one or more heteroatoms; each R′ independently represents hydrogen, is absent, or is selected from the group comprising optionally substituted C1-C10 alkyl, an C1-C10 alkyl-aryl, —NH2, —OR′, —OH, —CONH2, —CONR′2, —CNNR′2, —CSNR′2, —CONH—OR′, —CONH—NR′2, —NHCOR′, —NHCSR′, —NHCNR′, —NC(═O)R′, —NC(═O)OR′, —NC(═O)NR′, —NC(═S)OR′, —NC(═S)NR′, —SO2R′, —SOR′, —SR′, —SO2OR′, —SO2N(R′)2, —NR′NR′2, —NR′NR′—OR′, —NNR′, carbonyl, an C1-C10 alkyl-cycloalkyl, optionally substituted C3-C10 cycloalkyl, optionally substituted C3-C10 heterocyclyl, optionally substituted heteroaryl, optionally substituted aryl, or a combination thereof as allowed by valency; and wherein at least one R1 comprises an amine.

In one embodiment, at least one R1 is or comprises the substituent.

In one embodiment, the compound is characterized by a pKa between about 8 and about 10.

In one embodiment, at least one R1 is

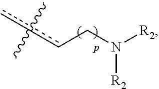

wherein p is between 1 and 5; and wherein each R2 is independently H, absent, or is selected from the group comprising an optionally substituted C1-C10 alkyl, an C1-C10 alkyl-aryl, an C1-C10 alkyl-cycloalkyl, optionally substituted C3-C10 cycloalkyl, optionally substituted C3-C10 heterocyclyl, optionally substituted heteroaryl, optionally substituted aryl, OR′, CONH2, CONR′2, CNNR′2, CSNR′2, CONH—OR′, CONH—NR′2, NHCOR′, NHCSR′, NHCNR′, —NC(═O)R′, —NC(═O)OR′, —NC(═O)NR′, —NC(═S)OR′, —NC(═S)NR′, SO2R′, SOR′, —SR′, SO2OR′, SO2N(R′)2, —NR′NR′2, —NR′NR′—OR′, —NNR′, carbonyl, C1-C10 haloalkyl, optionally substituted C1-C10 alkyl, NH2, NR′2, NH(C1-C10 alkyl), N(C1-C10 alkyl)2, C1-C10 haloalkoxy, hydroxy(C1-C10 alkyl), hydroxy(C1-C10 alkoxy), alkoxy(C1-C10 alkyl), alkoxy(C1-C10 alkoxy), amino(C1-C10 alkyl), CONH(C1-C10 alkyl), CON(C1-C10 alkyl)2, CO2H, CO2R′, —OCOR′, —C(═O)R′, —OC(═O)OR′, —OC(═O)NR′, —OC(═S)OR′, —OC(═S)NR′, a heteroatom, (C1-C10 alkyl)alkyl-cycloalkyl, (C1-C10 alkyl)alkyl-aryl, (C1-C10 alkyl)alkyl-heteroaryl, or a combination thereof as allowed by valency; or wherein both R2 are interconnected by a covalent bond, forming a 5-6 membered ring comprising one or more heteroatoms.

In one embodiment, the compound is represented by Formula 2:

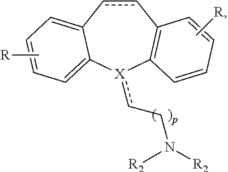

### or by Formula 2A:

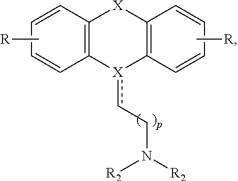

wherein each X is independently selected from C, CH, CH2, N, NR1, NH, O, and S.

In one embodiment, the compound is or comprises any one of:

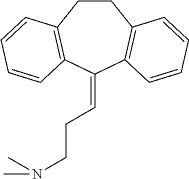 [SMILES: CN(C)CCC=C1c2ccccc2CCc2ccccc21]

including any salt, or any

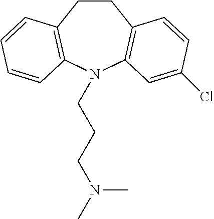 [SMILES: CN(C)CCCN1c2ccccc2CCc2ccc(Cl)cc21]

combination thereof.

In one embodiment, neutrophil-mediated inflammation

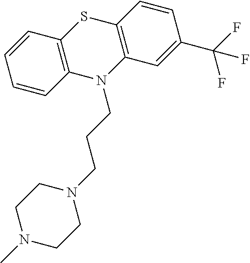 [SMILES: CN1CCN(CCCN2c3ccccc3Sc3ccc(C(F)(F)F)cc32)CC1]

comprises neutrophil-mediated tissue damage.

In one embodiment, the inflammation, tissue damage or both is mediated by ROS production by neutrophils.

In one embodiment, the inflammation, tissue damage or both comprises elevated liver enzyme levels, optionally wherein said liver enzyme is alanine aminotransferase (ALT).

In one embodiment, the subject suffers from a disease selected from an inflammatory disease, an autoimmune disease, cancer and an infectious disease.

In one embodiment, the disease is selected from acute respiratory distress syndrome (ARDS), chronic obstructive pulmonary disease (COPD), inflammatory bowel disease (IBD), sepsis, bacterial pneumonia, and viral pneumonia.

In one embodiment, the subject suffers from lung inflammation.

In one embodiment, the subject suffers from ARDS.

In another aspect, there is provided a pharmaceutical composition comprising a nanoparticle comprising an agent and a neutrophil targeting peptide; wherein said agent is a compound represented by Formula 1:

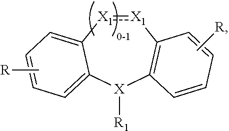

including any salt thereof; wherein: each R is independently H, or represents one or more substituents independently comprising any one of —NO₂, —CN, —OR′, —CONH₂, —CONR′₂, —CNNR′₂, —CSNR′₂, —CONH—OR′, —CONH—NR′₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NR′NR′₂, —NR′NR′—OR′, —NNR′, carbonyl, C₁-C₁₀ haloalkyl, optionally substituted C₁-C₁₀ alkyl, —NH₂, —NR′₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted, as allowed by valency; each X and X1 is independently selected from C, CR1, CH, CH2, N, NR1, NH, O, and S; each R1 is independently H, or

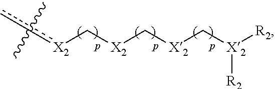

wherein each X₂ and X′₂ is independently selected from CR, CH, CH₂, N, NR, NH, O, and S, as allowed by valency, or is absent; optionally wherein any one of (i) two X2, (ii) two X′2 are interconnected by a covalent bond, forming a carbocyclic ring, optionally comprising one or more heteroatoms; p is between 0 and 10; each R2 is independently H, absent, or is selected from the group comprising an optionally substituted C1-C10 alkyl, an C1-C10 alkyl-aryl, an C1-C10 alkyl-cycloalkyl, optionally substituted C3-C10 cycloalkyl, optionally substituted C3-C10 heterocyclyl, optionally substituted heteroaryl, optionally substituted aryl, —NO2, —CN, —OR′, —CONH2, —CONR′2, —CNNR′2, —CSNR′2, —CONH—OR′, —CONH—NR′2, —NHCOR′, —NHCSR′, —NHCNR′, —NC(═O)R′, —NC(═O)OR′, —NC(═O)NR′, —NC(═S)OR′, —NC(═S)NR′, —SO2R′, —SOR′, —SR′, —SO2OR′, —SO2N(R′)2, —NR′NR′2, —NR′NR′—OR′, —NNR′, carbonyl, C1-C10 haloalkyl, optionally substituted C1-C10 alkyl, —NH2, —NR′2, —NH(C1-C10 alkyl), —N(C1-C10 alkyl)2, C1-C10 haloalkoxy, hydroxy(C1-C10 alkyl), hydroxy(C1-C10 alkoxy), alkoxy(C1-C10 alkyl), alkoxy(C1-C10 alkoxy), amino(C1-C10 alkyl), —CONH(C1-C10 alkyl), —CON(C1-C10 alkyl)2, —CO2H, —CO2R′, —OCOR′, —C(═O)R′, —OC(═O)OR′, —OC(═O)NR′, —OC(═S)OR′, —OC(═S)NR′, a heteroatom, (C1-C10 alkyl)alkyl-cycloalkyl, (C1-C10 alkyl)alkyl-aryl, (C1-C10 alkyl)alkyl-heteroaryl, or a combination thereof as allowed by valency; or wherein both R2 are interconnected by a covalent bond, forming a carbocyclic ring, optionally comprising one or more heteroatoms; each R′ independently represents hydrogen, is absent, or is selected from the group comprising optionally substituted C1-C10 alkyl, an C1-C10 alkyl-aryl, —NH2, —OR′, —OH, —CONH2, —CONR′2, —CNNR′2, —CSNR′2, —CONH—OR′, —CONH—NR′2, —NHCOR′, —NHCSR′, —NHCNR′, —NC(═O)R′, —NC(═O)OR′, —NC(═O)NR′, —NC(═S)OR′, —NC(═S)NR′, —SO2R′, —SOR′, —SR′, —SO2OR′, —SO2N(R′)2, —NR′NR′2, —NR′NR′—OR′, —NNR′, carbonyl, an C1-C10 alkyl-cycloalkyl, optionally substituted C3-C10 cycloalkyl, optionally substituted C3-C10 heterocyclyl, optionally substituted heteroaryl, optionally substituted aryl, or a combination thereof as allowed by valency; and wherein at least one R1 comprises an amine.

In one embodiment, at least one R1 is or comprises the substituent.

In one embodiment, the compound is characterized by a pKa between about 8 and about 10.

In one embodiment, at least one R1 is

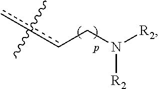

wherein p is between 1 and 5; and wherein each R2 is independently H, absent, or is selected from the group comprising an optionally substituted C1-C10 alkyl, an C1-C10 alkyl-aryl, an C1-C10 alkyl-cycloalkyl, optionally substituted C3-C10 cycloalkyl, optionally substituted C3-C10 heterocyclyl, optionally substituted heteroaryl, optionally substituted aryl, OR′, CONH2, CONR′2, CNNR′2, CSNR′2, CONH—OR′, CONH—NR′2, NHCOR′, NHCSR′, NHCNR′, —NC(═O)R′, —NC(═O)OR′, —NC(═O)NR′, —NC(═S)OR′, —NC(═S)NR′, SO2R′, SOR′, —SR′, SO2OR′, SO2N(R′)2, —NR′NR′2, —NR′NR′—OR′, —NNR′, carbonyl, C1-C10 haloalkyl, optionally substituted C1-C10 alkyl, NH2, NR′2, NH(C1-C10 alkyl), N(C1-C10 alkyl)2, C1-C10 haloalkoxy, hydroxy(C1-C10 alkyl), hydroxy(C1-C10 alkoxy), alkoxy(C1-C10 alkyl), alkoxy(C1-C10 alkoxy), amino(C1-C10 alkyl), CONH(C1-C10 alkyl), CON(C1-C10 alkyl)2, CO2H, CO2R′, —OCOR′, —C(═O)R′, —OC(═O)OR′, —OC(═O)NR′, —OC(═S)OR′, —OC(═S)NR′, a heteroatom, (C1-C10 alkyl)alkyl-cycloalkyl, (C1-C10 alkyl)alkyl-aryl, (C1-C10 alkyl)alkyl-heteroaryl, or a combination thereof as allowed by valency; or wherein both R2 are interconnected by a covalent bond, forming a 5-6 membered ring comprising one or more heteroatoms.

In one embodiment, the compound is represented by Formula 2:

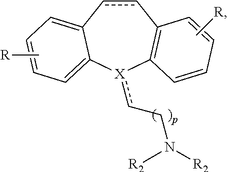

### or by Formula 2A:

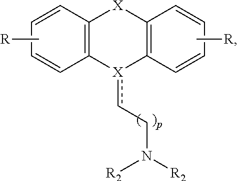

wherein each X is independently selected from C, CH, CH2, N, NR1, NH, O, and S.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-C: (1A) Line graph of ROS production over time from neutrophils. Neutrophils were either cultured alone (Cont.), with only PMA, or with PMA and one of seven tested compounds (A-G) at a concentration of 10 uM. (1B) Bar graph of % ROS production from the neutrophils of 1A as measured during the linear phase of ROS increase. ROS production by neutrophils exposed to only PMA was set as 100%. (1C) Line graph of ROS production over time from neutrophils contacted with only PMA or PMA and Palbociclib.

FIGS. 2A-B: Line graphs showing relative ROS levels from (2A) human and (2B) mouse neutrophils exposed to various compounds at various concentrations to establish IC50.

FIGS. 3A-D: (3A-B) Line graphs of ROS production over time from neutrophils. Neutrophils were culture (3A) without or (3B) with addition of PMA and the compounds at high and low concentrations. (3C-D) Bar graph of % ROS production from the control neutrophils of (3C) 3A and PMA treated neutrophils (3D) 3B as measured during the linear phase of ROS increase. ROS production by neutrophils not treated with any of the compounds was set as 100%.

FIGS. 4A-B: (4A) Bar graph of relative neutrophil death as measured by double positive Annexin V and PI staining in isolated neutrophils cultured for 2 hours with various compounds at a concentration of 1 uM. (4B) Bar graph of % bacterial killing in blood treated with the various compounds of the invention.

FIGS. 5A-D: (5A) Bar graph of Cy5 levels in inflamed lungs after IV administration of naked liposomes encapsulating Cy5 and targeting peptide decorated liposomes encapsulating Cy5. (5B) Bar graph of relative ROS levels in the lungs of LPS treated mice, LPS treated mice administered free Stelazine, Stelazine loaded naked liposomes and Stelazine loaded targeting-peptide decorated liposomes. (5C) Images of ROS levels in LPS treated lungs administered various compositions of Stelazine and/or naked or peptide-decorated liposomes (referred to as “TENN”). 4 hours following treatment with the described molecules, animals were injected with luminol and ROS in the lungs was quantified using IVIS. (5D) Bar graph of circulating ALT levels 48 hours after drug administration in control mice (untreated), mice treated with free Stelazine and mice treated with neutrophil targeted liposomes containing Stelazine.

FIGS. 6A-J are chemical structures of exemplary compounds disclosed herein: (6A) represents a chemical structure of Abemaciclib. (6B) represents a chemical structure of Palbociclib. (6C) represents a chemical structure of Ribociclib. (6D) represents a chemical structure of Bosutinib. (6E) represents a chemical structure of Afatinib. (6F) represents a chemical structure of Trifluoperazine. (6G) represents a chemical structure of Dyclonine. (6H) represents a chemical structure of Clomipramine. (6I) represents a chemical structure of Amitriptyline. (6J) represents chemical structures of additional molecules which inhibited neutrophil ROS.

### DETAILED DESCRIPTION OF THE INVENTION

The present invention, in some embodiments, provides methods of inhibiting reactive oxygen species (ROS) production by neutrophils. Methods of treating neutrophil-mediated inflammation are also provided. The present invention further concerns pharmaceutical composition comprising a nanoparticle comprising a neutrophil targeting peptide and an agent that inhibits neutrophil ROS production.

By a first aspect, there is provided a method of inhibiting reactive oxygen species (ROS) production by neutrophils, the method comprising contacting the neutrophils with a ROS-inhibiting agent

By another aspect, there is provided a method of inhibiting reactive oxygen species (ROS) production by neutrophils, the method comprising contacting the neutrophils with an agent, wherein the agent is a cyclin dependent kinase 4 (CDK4) inhibitor.

By another aspect, there is provided a method of inhibiting reactive oxygen species (ROS) production by neutrophils, the method comprising contacting the neutrophils with an agent, wherein the agent is a cyclin dependent kinase 6 (CDK6) inhibitor.

By another aspect, there is provided a method of inhibiting reactive oxygen species (ROS) production by neutrophils, the method comprising contacting the neutrophils with an agent, wherein the agent is an epidermal growth factor receptor (EGFR) inhibitor.

By another aspect, there is provided a method of inhibiting reactive oxygen species (ROS) production by neutrophils, the method comprising contacting the neutrophils with an agent, wherein the agent is a proto-oncogene tyrosine-protein kinase Src (SRC) inhibitor.

By another aspect, there is provided a method of inhibiting reactive oxygen species (ROS) production by neutrophils, the method comprising contacting the neutrophils with an agent, wherein the agent is a sodium (Na) channel blocker.

By another aspect, there is provided a method of inhibiting reactive oxygen species (ROS) production by neutrophils, the method comprising contacting the neutrophils with a compound, wherein the compound is represented by Formula 1:

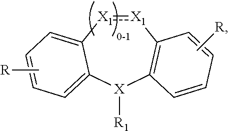

including any salt thereof.

By another aspect, there is provided a method of treating neutrophil-mediated inflammation or a neutrophil-associated disease or condition in a subject, the method comprising administering to the subject a ROS inhibiting agent, thereby treating neutrophil-mediated inflammation in a subject.

By another aspect, there is provided a method of treating neutrophil-mediated inflammation or a neutrophil-associated disease or condition in a subject, the method comprising administering to the subject an agent, wherein the agent is a CDK4 inhibitor, thereby treating neutrophil-mediated inflammation in a subject.

By another aspect, there is provided a method of treating neutrophil-mediated inflammation or a neutrophil-associated disease or condition in a subject, the method comprising administering to the subject an agent, wherein the agent is a CDK6 inhibitor, thereby treating neutrophil-mediated inflammation in a subject.

By another aspect, there is provided a method of treating neutrophil-mediated inflammation or a neutrophil-associated disease or condition in a subject, the method comprising administering to the subject an agent, wherein the agent is an EGFR inhibitor, thereby treating neutrophil-mediated inflammation in a subject.

By another aspect, there is provided a method of treating neutrophil-mediated inflammation or a neutrophil-associated disease or condition in a subject, the method comprising administering to the subject an agent, wherein the agent is a SRC inhibitor, thereby treating neutrophil-mediated inflammation in a subject.

By another aspect, there is provided a method of treating neutrophil-mediated inflammation or a neutrophil-associated disease or condition in a subject, the method comprising administering to the subject an agent, wherein the agent is a Na channel blocker, thereby treating neutrophil-mediated inflammation in a subject.

By another aspect, there is provided a method of treating neutrophil-mediated inflammation or a neutrophil-associated disease or condition in a subject, the method comprising administering to the subject a compound, wherein the compound is represented by Formula 1:

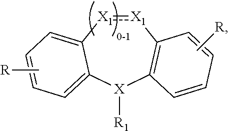

R1, including any salt thereof.

By another aspect, there is provided a CDK4 inhibitor for use in treating neutrophil-mediated inflammation or a neutrophil-associated disease or condition.

By another aspect, there is provided a CDK6 inhibitor for use in treating neutrophil-mediated inflammation or a neutrophil-associated disease or condition.

By another aspect, there is provided an EGFR inhibitor for use in treating neutrophil-mediated inflammation or a neutrophil-associated disease or condition.

By another aspect, there is provided a SRC inhibitor for use in treating neutrophil-mediated inflammation or a neutrophil-associated disease or condition.

By another aspect, there is provided a Na channel blocker for use in treating neutrophil-mediated inflammation or a neutrophil-associated disease or condition.

By another aspect, there is provided a compound for use in treating neutrophil-mediated inflammation or a neutrophil-associated disease or condition, wherein the compound is represented by Formula 1:

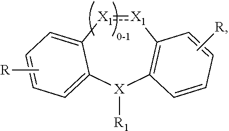

including any salt thereof.

The term “neutrophil” as used herein refers to the type of leucocyte most numerous in mammals, which forms an important part of the innate immune system. Neutrophils form part of a family of polymorphonuclear cells (PMN) with basophils and eosinophils. Neutrophils are normally found in the bloodstream. During the starting (acute) phase of inflammation, particularly as a result of bacterial infection, and certain forms of cancer, neutrophils are among the first immune cells migrating towards the site of inflammation/tumor. They migrate through the blood vessels, then through interstitial tissue, following chemical signals, such as interleukin-8 (IL-8) and C5a. Neutrophils can be mature, immature, high-density or low-density, including but not limited to High Density Neutrophils—HDNs and Low Density Neutrophils (LDNs).

In some embodiments, the neutrophils are mammalian neutrophils. In some embodiments, the mammal is a rodent. In some embodiments, the rodent is a mouse. In some embodiments, the neutrophils are murine neutrophils. In some embodiments, the mammal is human. In some embodiments, the neutrophils are human neutrophils. In some embodiments, the neutrophils are ex vivo. In some embodiments, the neutrophils are in vitro. In some embodiments, the neutrophils are in vivo. In some embodiments, the neutrophils are in a subject. In some embodiments, the neutrophils are circulating neutrophils. In some embodiments, the neutrophils are tissue resident neutrophils. In some embodiments, the neutrophils are neutrophils at a site of inflammation. In some embodiments, the neutrophils are at a site of infection. In some embodiments, the neutrophils are in the lung. In some embodiments, the neutrophils are in the liver. In some embodiments, the neutrophils are in the digestive tract. In some embodiments, the neutrophils are in the bowl. In some embodiments, the neutrophils are in a tumor. In some embodiments, the neutrophils are tumor associated neutrophils (TANs). In some embodiments, the neutrophils are myeloid derived suppressor cells (MDSCs). In some embodiments, the inflammation is autoimmune inflammation.

In some embodiments, the method is an in vitro method. In some embodiments, the method is an ex vivo method. In some embodiments, the method is an in vivo method. In some embodiments, the neutrophils are contacted ex vivo and then returned to the body of the subject.

As used herein, the term “reactive oxygen species” refers to highly reactive chemicals formed from O2. Examples of ROS include, but are not limited to peroxides, superoxides, hydroxyl radicals, singlet oxygen and alpha-oxygen. ROSs are generally a byproduct of oxygen metabolism and are generally cytotoxic as they can damage the cellular membrane. Neutrophils use ROS to damage foreign cells as part of the innate immune response. In some embodiments, the ROS is hydrogen peroxide. In some embodiments, the ROS is intracellular ROS. In some embodiments, the ROS is intercellular ROS. In some embodiments, the ROS is Phorbol 12-myristate 13-acetate (PMA) stimulated ROS. In some embodiments, the ROS is N-Formylmethionine-leucyl-phenylalanine (fMLP) stimulated ROS. Methods of measuring ROS and ROS production are well known in the art and include for example ROS detection kits and assays (including luminol based assays) such as are commercially available and well-known in the art.

In some embodiments, the method comprises contacting a neutrophil with an agent. In some embodiments, the agent is a drug. In some embodiments, the drug is an FDA approved drug. In some embodiments, the agent is a compound. In some embodiments, the compound is represented by Formula 1. In some embodiments, Formula 1 comprises a salt of a compound of Formula 1. In some embodiments, the agent is a ROS inhibiting agent. In some embodiments, the agent is a neutrophil ROS inhibiting agent. In some embodiments, the agent inhibits ROS production. In some embodiments, the agent inhibits ROS secretion. In some embodiments, the agent inhibits ROS delivery to the outside of neutrophils. In some embodiments, outside the neutrophils is into serum. In some embodiments, outside the neutrophils is into blood. In some embodiments, outside the neutrophils is into tissue. In some embodiments, the neutrophils are activated neutrophils. In some embodiments, the agent inhibits ROS production by activated neutrophils.

In some embodiments, the agent does not induce neutrophil death. In some embodiments, the agent does not substantially induce neutrophil death. In some embodiments, the agent is not toxic to neutrophils. In some embodiments, the inhibition in ROS production is not due to neutrophil death. In some embodiments, the agent does not induce neutropenia. In some embodiments, the agent does not induce neutropenia in a subject administered the agent.

In some embodiments, the agent is a single agent. In some embodiments, the agent is multiple agents. In some embodiments, the agent is a combination of agents. In some embodiments, the agent is a cocktail of agents. In some embodiments, the agent is a plurality of agents. In some embodiments, a plurality is 2 agents. In some embodiments, a plurality is 4 agents. In some embodiments, the plurality comprises Bosutinib, Afatanib, Clomipramine and Stelazine. In some embodiments, the plurality consists Bosutinib, Afatanib, Clomipramine and Stelazine.

In some embodiments, the agent is a small molecule. In some embodiments, the agent is a small molecule inhibitor. In some embodiments, an inhibitor is a blocker. In some embodiments, the agent is a direct inhibitor. In some embodiments, the agent is an indirect inhibitor. As used herein, a “direct inhibitor” refers to a molecule that physically contacts the molecule it targets are causes inhibition. As used herein, an “indirect inhibitor” is a molecule that causes inhibition but does not directly contact the inhibited target. In some embodiments, the inhibitor is a specific inhibitor. As used herein, a “specific inhibitor” is a molecule that only effects or only significantly effects a given target. A specific inhibitor can be direct or indirect. In some embodiments, the mechanism of inhibition is not known. In some embodiments, the inhibitor is an ATP-competitive inhibitor. In some embodiments, the agent is a small molecule. In some embodiments, the agent is a small molecule inhibitor. In some embodiments, the agent is a monoclonal antibody.

In some embodiments, the agent is a cyclin dependent kinase (CDK) inhibitor. In some embodiments, the agent is a pan CDK inhibitor. In some embodiments, the agent is a CDK4 inhibitor. In some embodiments, the agent is a CDK6 inhibitor. In some embodiments, the agent is a CDK4/6. In some embodiments, the agent is a dual CDK4/6 inhibitor. In some embodiments, the agent is both a CDK4 and a CDK6 inhibitor. In some embodiments, the inhibitor specifically inhibits CDK4. In some embodiments, the inhibitor specifically inhibits CDK6. In some embodiments, the inhibitor specifically inhibits CDK4/6. In some embodiments, the inhibitor directly inhibits CDK4. In some embodiments, the inhibitor directly inhibits CDK6. In some embodiments, the inhibitor directly inhibits CDK4/6.

CDK4 and/or CDK6 inhibitors are well known in the art and example include but are not limited to, Abemaciclib, Ribociclib, Palbociclib, Trilaciclib, Lerociclib, SHR-6390, BPI-16350 and P1446A-05. Only Abemaciclib, Ribociclib, and Palbociclib have been FDA approved. Other examples of potential CDK4/6 inhibitors can be found in Ammazzalorso et al., 2021 “Development of CDK4/6 inhibitors: A five years update” Molecules, 26, 1488, herein incorporated by reference in its entirety. In some embodiments, the CDK4/6 inhibitor is Abemaciclib. In some embodiments, the CDK4/6 inhibitor is Ribociclib. In some embodiments, the CDK4/6 inhibitor is Palbociclib. In some embodiments, the CDK4/6 inhibitor is selected from Abemaciclib, Ribociclib, and Palbociclib. In some embodiments, the CDK4/6 inhibitor is selected from Abemaciclib, and Ribociclib. In some embodiments, the agent is Abemaciclib. In some embodiments, the agent is Ribociclib. In some embodiments, the agent is Palbociclib.

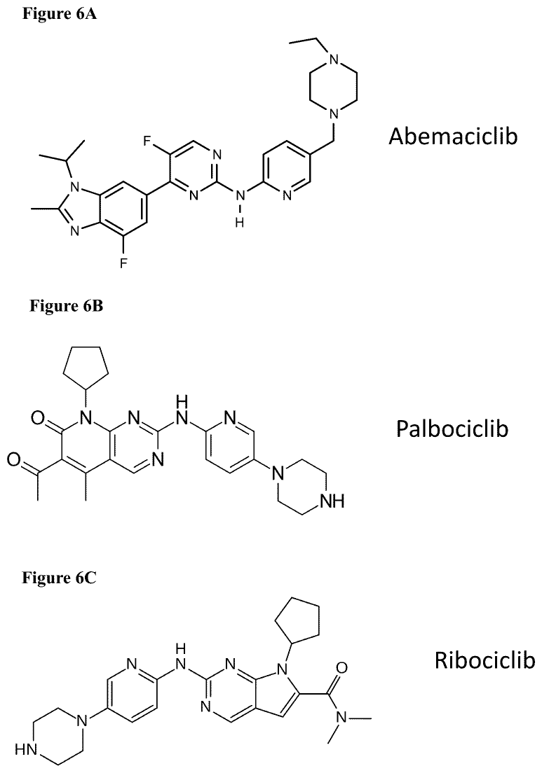

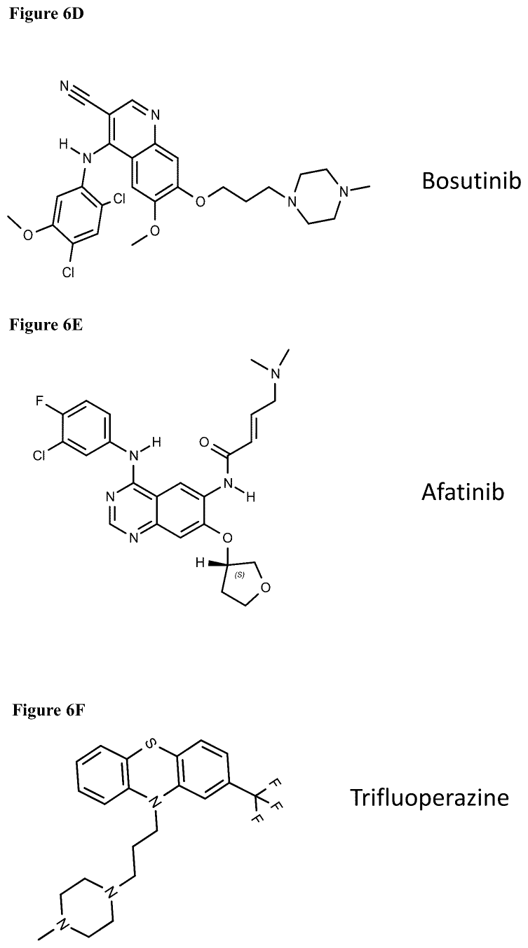

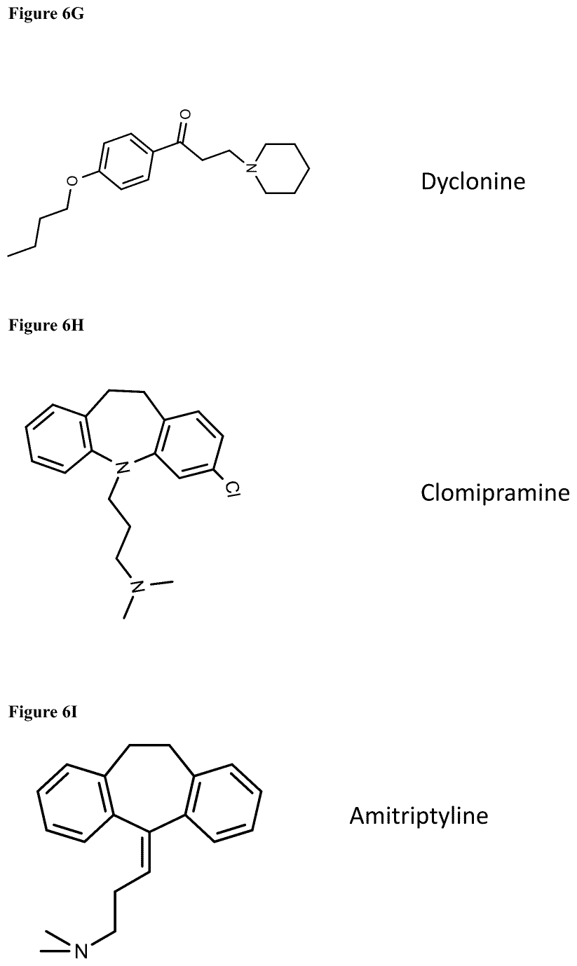

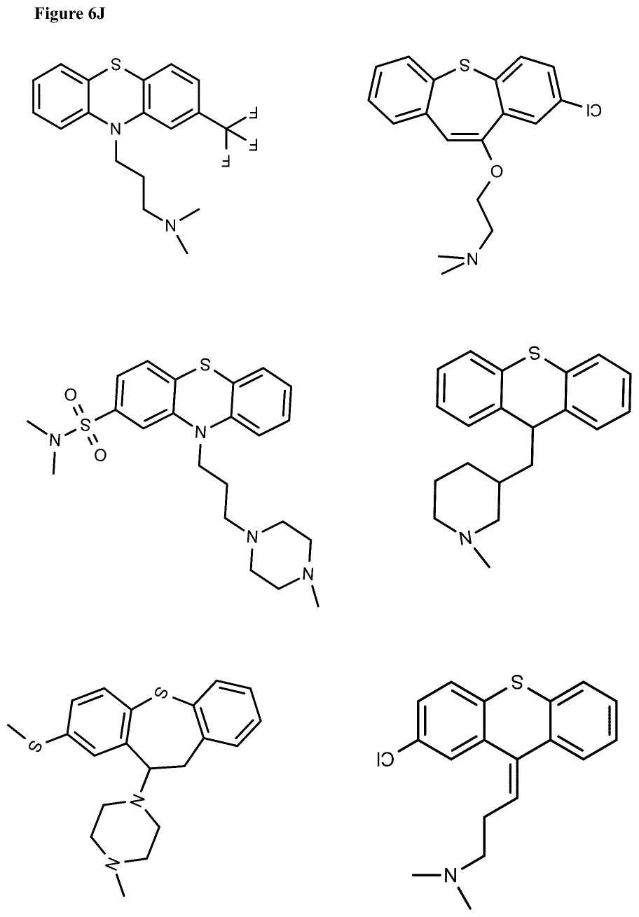

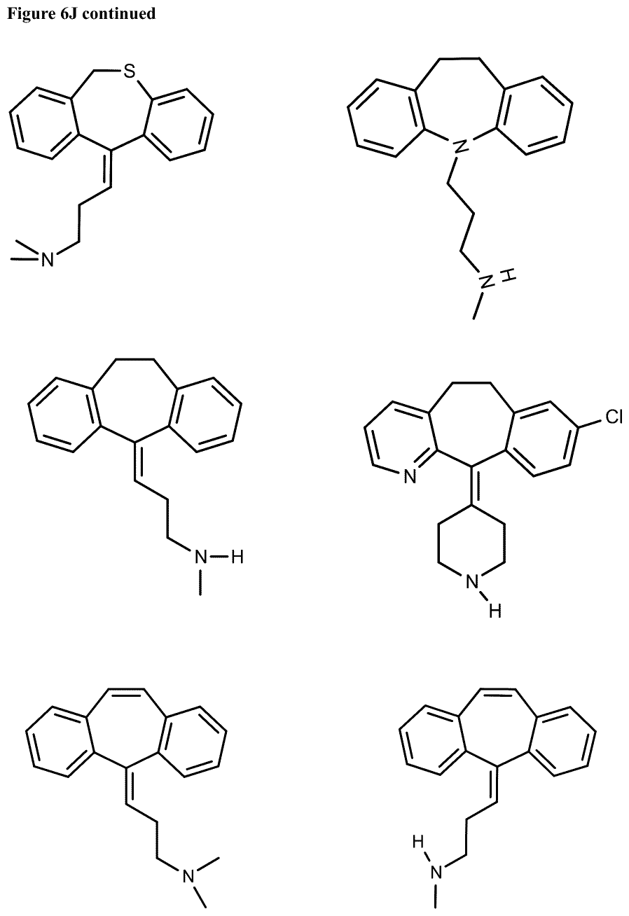

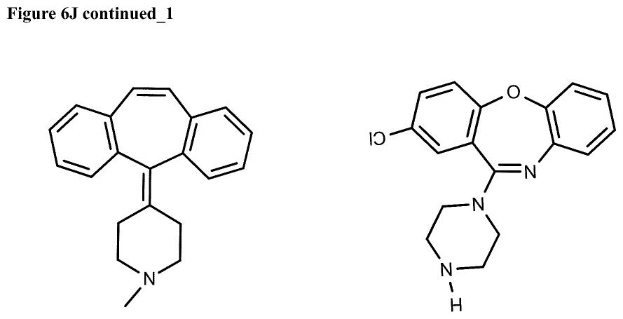

Abemaciclib, which is sold under the brand name Verzenio®, is a well-known medication for the treatment of metastatic breast cancer and possesses the molecular formula presented in FIG. 6A. Abemaciclib is a dual selective inhibitor of CDK4 and CDK6. Palbociclib and Ribociclib are closely related to Abemaciclib and are both also dual selective inhibitors of CDK4 and CDK6. Palbociclib is sold under the brand name Ibrance® and its molecular formula is provided in FIG. 6B. Ribociclib is sold under the brand names Kisqali® and Kryxana® and its molecular formula is provided in FIG. 6C.

In some embodiments, the agent inhibits a target upstream to CDK4/6. In some embodiments, upstream of CDK4/6 is upstream of CDK4/6 activation. In some embodiments, the target when active induces CDK4/6 activation. In some embodiments, the target when active induces CDK4/6 expression. In some embodiments, an agent that inhibits an upstream target is an indirect inhibitor of CDK4/6. In some embodiments, upstream target is SRC. In some embodiments, the upstream target is EGFR.

In some embodiments, the agent is a SRC inhibitor. In some embodiments, a SRC inhibitor is a SRC tyrosine kinase inhibitor. In some embodiments, the inhibitor is a tyrosine kinase inhibitor. In some embodiments, the agent is a direct inhibitor of SRC. In some embodiments, the agent inhibits a kinase function of SRC. In some embodiments, the inhibitor inhibits hyperactive SRC. In some embodiments, the inhibitor specifically inhibits SRC. In some embodiments, a SRC inhibitor is a SRC, LYN and HCK inhibitor. In some embodiments, the inhibitor directly inhibits SRC. In some embodiments, a SRC inhibitor inhibits the SRC pathway. SRC inhibitors are well known in the art and include for non-limiting example Bosutinib, Saracatinib, KX2-391, PP1, PP2 and Dasatinib. In some embodiments, the SRC inhibitor is Bosutinib. In some embodiments, SRC inhibitor is also a BCR-ABL tyrosine kinase inhibitor.

Bosutinib, which is sold under the brand name Bosulif®, is a well-known medication for the treatment of chronic myelogenous leukemia (CML) and possesses the molecular formula presented in FIG. 6D. Bosutinib is known to inhibit both BCR-ABL and SRC.

In some embodiments, the agent is an EGFR inhibitor. In some embodiments, the agent is a direct inhibitor of EGFR. In some embodiments, the inhibitor is a specific inhibitor of EGFR. In some embodiments, the inhibitor inhibits EGFR signaling. In some embodiments, the inhibitor inhibits binding of EGF to EGFR. In some embodiments, the inhibitor inhibits EGFR tyrosine kinase function. In some embodiments, the EGFR tyrosine kinase function comprises EGFR mediated phosphorylation. In some embodiments, the inhibitor is a tyrosine kinase inhibitor. In some embodiments, the inhibitor is a small molecule inhibitor. In some embodiments, the inhibitor is a monoclonal antibody inhibitor. EGFR inhibitors are well-known in the art and include for non-limiting example, Afatinib, Trifluoperazine, Erlotinib, Gefitinib, Lapatinib, Osimertinib, Neratinib, Dacomitinib, Vandetanib, cetuximab, panitumumab, and necitumumab. In some embodiments, the EGFR inhibitor is Afatinib. In some embodiments, the EGFR inhibitor is Trifluoperazine.

Afatinib, which is sold under the brand name Gilotrif®, is a well-known medication for the treatment of non-small cell lung carcinoma (NSCLC) and possesses the molecular formula presented in FIG. 6E. Afatinib is known inhibitor of EGFR and is predominantly used to treat NSCLCs that harbor EGFR mutations. Afatinib is also used as a salt, such as Afatinib Maleate.

Trifluoperazine, which is sold under the brand name Stelazine®, is a well-known antipsychotic medication used for the treatment of schizophrenia and possesses the molecular formula presented in FIG. 6F. Trifluoperazine has been shown to inhibit EGFR and specifically EGFR mediated phosphorylation (see Ross et al., “Inhibition of epidermal growth factor-induced phosphorylation by trifluoperazine”, 1985, J. Cell Physiol., September; 124(3):499-506, herein incorporated by reference in its entirety). Further, Trifluoperazine has also been shown to be a direct sodium channel blocker whose blockade is not mediated by calmodulin (see Kim et al., “Trifluoperazine blocks the human cardiac sodium channel, Nav 1.5, independent of calmodulin”, 2016, Biochem. Biophys. Res Commun., October 21; 479(3):584-589, herein incorporated by reference in its entirety).

In some embodiments, the agent is a Na channel blocker. In some embodiments, the agent is a Na channel inhibitor. In some embodiments, a blocker is an inhibitor. In some embodiments, a blocker blocks channel opening. In some embodiments, an inhibitor inhibits channel function. In some embodiments, channel function comprises sodium influx. In some embodiments, channel function comprises sodium efflux. In some embodiments, influx and/or efflux are through a cellular membrane. In some embodiments, the sodium channel is a voltage-gated sodium channel. Na channel blockers are well-known in the art and include for non-limiting example Dyclonine, Trifluoperazine, Clomipramine, Amitriptyline, Acetylprocainamide, Dibucaine, Prilocaine, Proparacine, Quinidine, Tetracaine, Moricizine, Oxcarbazepine, Quinidine, Procainamide, Disopyramide, Lidocaine, Mexiletine, Tocainide, Phenytoin, Encainide, Flecainide, and Propafenone. In some embodiments, the Na channel blocker is also an anesthetic. In some embodiments, an anesthetic is an oral anesthetic. In some embodiments, the Na channel blocker is Dyclonine. In some embodiments, the Na channel blocker is Trifluoperazine. In some embodiments, the Na channel blocker is a tricyclic antidepressant (TCA). In some embodiments, the TCA is selected from Clomipramine, and Amitriptyline. In some embodiments, the Na channel blocker is Clomipramine. In some embodiments, the Na channel blocker is Amitriptyline. In some embodiments, the agent is a Na channel blocker and an EGFR inhibitor.

Dyclonine is a well-known local anesthetic and is the active ingredient in over-the-counter sore throat treatments. It possesses the molecular formula presented in FIG. 6G. In addition to its analgesic properties, Dyclonine is a well-known Na channel blocker. Dyclonine is also used as a salt such as Dyclonine HCl.

Clomipramine and Amitriptyline are well-known TCAs. TCAs are so call because their chemical structure contains three rings. Though the rings generally contain carbons, nitrogens, serines and oxygens can also be present. Clomipramine, which is sold under the brand name Anafranil®, is a well-known antidepressant used for the treatment of OCD, major depressive disorder and other neurological conditions. It possesses the molecular formula presented in FIG. 6H. Amitriptyline, which is sold under the brand name Elavil®, is also a well-known antidepressant used for the treatment of cyclic vomiting syndrome, major depressive disorder and other neurological conditions. It possesses the molecular formula presented in FIG. 6I. TCAs are known to be Na channel blockers and a wide variety of TCAs have been shown to block sodium channels.

Although Trifluoperazine is not a TCA, as it is not considered an antidepressant, it shares the tricyclic structure of TCAs. A total of 17 tricyclic molecules were found in the ROS screen performed hereinbelow (of the 160 total hits). In addition to Trifluoperazine, Clomipramine, and Amitriptyline the other 14 compounds are presented in FIG. 6J.

In some embodiments, the agent is selected from a cyclin dependent kinase 4 (CDK4) inhibitor, a cyclin dependent kinase 6 (CDK6) inhibitor, an epidermal growth factor receptor (EGFR) inhibitor, a proto-oncogene tyrosine-protein kinase Src (SRC) inhibitor, a sodium (Na) channel blocker and a combination thereof. In some embodiments, the agent is selected from a cyclin dependent kinase 4 (CDK4) inhibitor, a cyclin dependent kinase 6 (CDK6) inhibitor, an epidermal growth factor receptor (EGFR) inhibitor, a proto-oncogene tyrosine-protein kinase Src (SRC) inhibitor, and a sodium (Na) channel blocker. In some embodiments, the agent is selected from a cyclin dependent kinase 4 (CDK4) inhibitor, a cyclin dependent kinase 6 (CDK6) inhibitor, an epidermal growth factor receptor (EGFR) inhibitor, and a proto-oncogene tyrosine-protein kinase Src (SRC) inhibitor. In some embodiments, the agent is selected from a cyclin dependent kinase 4 (CDK4) inhibitor, a cyclin dependent kinase 6 (CDK6) inhibitor, and an epidermal growth factor receptor (EGFR) inhibitor. In some embodiments, the agent is selected from a cyclin dependent kinase 4 (CDK4) inhibitor, a cyclin dependent kinase 6 (CDK6) inhibitor, an epidermal growth factor receptor (EGFR) inhibitor, and a sodium (Na) channel blocker. In some embodiments, the agent is selected from a cyclin dependent kinase 4 (CDK4) inhibitor, a cyclin dependent kinase 6 (CDK6) inhibitor, and a sodium (Na) channel blocker. In some embodiments, the agent is selected from a cyclin dependent kinase 4 (CDK4) inhibitor, a cyclin dependent kinase 6 (CDK6) inhibitor, a proto-oncogene tyrosine-protein kinase Src (SRC) inhibitor, and a sodium (Na) channel blocker. In some embodiments, the agent is selected from a cyclin dependent kinase 4 (CDK4) inhibitor, a cyclin dependent kinase 6 (CDK6) inhibitor, and a sodium (Na) channel blocker.

In some embodiments, the agent is a compound represented by Formula 1 or any salt thereof. In some embodiments, the EGFR inhibitor comprises a compound represented by Formula 1 or any salt thereof. In some embodiments, the Na channel blocker comprises a compound represented by Formula 1 or any salt thereof.

In some embodiments, each R is independently H, or represents one or more substituents independently comprising any one of NO2, CN, OR′, CONH2, CONR′2, CNNR′2, CSNR′2, CONH—OR′, CONH—NR′2, NHCOR′, NHCSR′, NHCNR′, —NC(═O)R′, —NC(═O)OR′, —NC(═O)NR′, —NC(═S)OR′, —NC(═S)NR′, SO2R′, SOR′, —SR′, SO2OR′, SO2N(R′)2, —NR′NR′2, —NR′NR′—OR′, —NNR′, carbonyl, C1-C10 haloalkyl, optionally substituted C1-C10 alkyl, NH2, NR′2, NH(C1-C10 alkyl), N(C1-C10 alkyl)2, C1-C10 haloalkoxy, hydroxy(C1-C10 alkyl), hydroxy(C1-C10 alkoxy), alkoxy(C1-C10 alkyl), alkoxy(C1-C10 alkoxy), amino(C1-C10 alkyl), CONH(C1-C10 alkyl), CON(C1-C10 alkyl)2, CO2H, CO2R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C1-C10 alkyl)alkyl-cycloalkyl, (C1-C10 alkyl)alkyl-aryl, (C1-C10 alkyl)alkyl-heteroaryl, or any combination thereof, and wherein each of cycloalkyl, heterocyclyl aryl, heteroaryl is substituted or non-substituted, as allowed by valency.

In some embodiments, each X and X1 is independently selected from C, CR1, CH, CH2, N, NR1, NH, O, and S. In some embodiments, p is between 0 and 10.

In some embodiments, each R1 is independently H, or

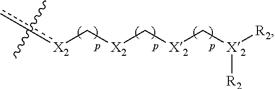

wherein each X₂ and X′₂ is independently selected from CR, CH, CH2, N, NR, NH, O, and S, as allowed by valency, or is absent. In some embodiments, any one of (i) two X2, (ii) two X′2 are interconnected by a covalent bond, forming a carbocyclic ring. In some embodiments, the carbocyclic ring comprises one or more heteroatoms.

In some embodiments, each R2 is independently H, absent, or is selected from the group comprising an optionally substituted C1-C10 alkyl, an C1-C10 alkyl-aryl, an C1-C10 alkyl-cycloalkyl, optionally substituted C3-C10 cycloalkyl, optionally substituted C3-C10 heterocyclyl, optionally substituted heteroaryl, optionally substituted aryl, —NO2, —CN, —OR′, —CONH2, —CONR′2, —CNNR′2, —CSNR′2, —CONH—OR′, —CONH—NR′2, —NHCOR′, —NHCSR′, —NHCNR′, —NC(═O)R′, —NC(═O)OR′, —NC(═O)NR′, —NC(═S)OR′, —NC(═S)NR′, —SO2R′, —SOR′, —SR′, —SO2OR′, —SO2N(R′)2, —NR′NR′2, —NR′NR′—OR′, —NNR′, carbonyl, C1-C10 haloalkyl, optionally substituted C1-C10 alkyl, —NH2, —NR′2, —NH(C1-C10 alkyl), —N(C1-C10 alkyl)2, C1-C10 haloalkoxy, hydroxy(C1—C10 alkyl), hydroxy(C1-C10 alkoxy), alkoxy(C1-C10 alkyl), alkoxy(C1-C10 alkoxy), amino(C1-C10 alkyl), —CONH(C1-C10 alkyl), —CON(C1-C10 alkyl)2, —CO2H, —CO2R′, —OCOR′, —C(═O)R′, —OC(═O)OR′, —OC(═O)NR′, —OC(═S)OR′, —OC(═S)NR′, a heteroatom, (C1-C10 alkyl)alkyl-cycloalkyl, (C1-C10 alkyl)alkyl-aryl, (C1-C10 alkyl)alkyl-heteroaryl, or a combination thereof as allowed by valency. In some embodiments, both R2 are interconnected by a covalent bond, forming a carbocyclic ring. In some embodiments, the carbocyclic ring comprises one or more heteroatoms.

In some embodiments, each R′ independently represents hydrogen, is absent, or is selected from the group comprising optionally substituted C1-C10 alkyl, an C1-C10 alkyl-aryl, —NH2, —OR′, —OH, —CONH2, —CONR′2, —CNNR′2, —CSNR′2, —CONH—OR′, —CONH—NR′2, —NHCOR′, —NHCSR′, —NHCNR′, —NC(═O)R′, —NC(═O)OR′, —NC(═O)NR′, —NC(═S)OR′, —NC(═S)NR′, —SO2R′, —SOR′, —SR′, —SO2OR′, —SO2N(R′)2, —NR′NR′2, —NR′NR′—OR′, —NNR′, carbonyl, an C1-C10 alkyl-cycloalkyl, optionally substituted C3-C10 cycloalkyl, optionally substituted C3-C10 heterocyclyl, optionally substituted heteroaryl, optionally substituted aryl, or a combination thereof as allowed by valency.

In some embodiments, at least one R1 comprises an amine. In some embodiments, at least one R1 is the substituent. In some embodiments, at least one R1 comprises the substituent. In some embodiments, at least one R1 is

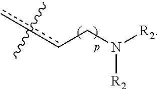

In some embodiments, p is between 1 and 5. In some embodiments, each R2 is independently H, absent, or is selected from the group comprising an optionally substituted C1-C10 alkyl, an C1-C10 alkyl-aryl, an C1-C10 alkyl-cycloalkyl, optionally substituted C3-C10 cycloalkyl, optionally substituted C3-C10 heterocyclyl, optionally substituted heteroaryl, optionally substituted aryl, OR′, CONH2, CONR′2, CNNR′2, CSNR′2, CONH—OR′, CONH—NR′2, NHCOR′, NHCSR′, NHCNR′, —NC(═O)R′, —NC(═O)OR′, —NC(═O)NR′, —NC(═S)OR′, —NC(═S)NR′, SO2R′, SOR′, —SR′, SO2OR′, SO2N(R′)2, —NR′NR′2, —NR′NR′—OR′, —NNR′, carbonyl, C1-C10 haloalkyl, optionally substituted C1-C10 alkyl, NH2, NR′2, NH(C1-C10 alkyl), N(C1-C10 alkyl)2, C1-C10 haloalkoxy, hydroxy(C1-C10 alkyl), hydroxy(C1-C10 alkoxy), alkoxy(C1-C10 alkyl), alkoxy(C1-C10 alkoxy), amino(C1-C10 alkyl), CONH(C1-C10 alkyl), CON(C1-C10 alkyl)2, CO2H, CO2R′, —OCOR′, —C(═O)R′, —OC(═O)OR′, —OC(═O)NR′, —OC(═S)OR′, —OC(═S)NR′, a heteroatom, (C1-C10 alkyl)alkyl-cycloalkyl, (C1-C10 alkyl)alkyl-aryl, (C1-C10 alkyl)alkyl-heteroaryl, or a combination thereof as allowed by valency. In some embodiments, both R2 are interconnected by a covalent bond, forming a 5-6 membered ring comprising one or more heteroatoms.

In some embodiments, the compound is characterized by a pKa between about 6 and about 12, about 6 and about 11, about 6 and about 10, about 7 and about 12, about 7 and about 11, about 7 and about 10, about 8 and about 12, about 8 and about 11, about 8 and about 10, about 9 and about 12, about 9 and about 11, or about 9 and about 10. Each possibility represents a separate embodiment of the invention. In some embodiments, the compound is characterized by a pKa between about 8 and about 10.

In some embodiments, the compound is represented by Formula 2:

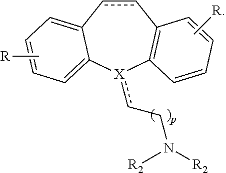

In some embodiments, the compound is represented by Formula 2A:

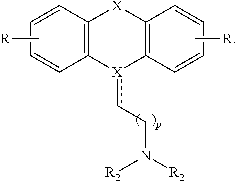

In some embodiments, the compound represented by Formula 1 is a compound represented by Formula 2. In some embodiments, the compound represented by Formula 1 is a compound represented by Formula 2A. In some embodiments, each X is independently selected from C, CH, CH2, N, NR1, NH, O, and S.

In some embodiments, the compound is Trifluoperazine. In some embodiments, the compound is Trifluoperazine or a salt thereof. In some embodiments, the compound is the compound provided in FIG. 6F. In some embodiments, the compound is Clomipramine. In some embodiments, the compound is Clomipramine or a salt thereof. In some embodiments, the compound is the compound provided in FIG. 6H. In some embodiments, the compound is Amitriptyline. In some embodiments, the compound is Amitriptyline or a salt thereof. In some embodiments, the compound is the compound provided in FIG. 6I. In some embodiments, the compound is a compound provided in FIG. 6J. In some embodiments, the compound is a salt of a compound provided in FIG. 6J. In some embodiments, the compound is selected from Trifluoperazine, Clomipramine, Amitriptyline and a salt thereof.

In some embodiments, a salt of the compound disclosed herein refers to a pharmaceutically acceptable salt. As used herein, the term “pharmaceutically acceptable salt” refers to any non-toxic salt of a compound of the present invention that, upon administration to a subject, e.g., a human, is capable of providing, either directly or indirectly, a compound of this invention or an inhibitorily active metabolite or residue thereof. For example, the term “pharmaceutically acceptable” can mean approved by a regulatory agency of the Federal or a state government or listed in the U. S. Pharmacopeia or other generally recognized pharmacopeia for use in animals, and more particularly in humans.

Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified compound in its free-based form with a suitable organic or inorganic acid and 2) isolating the salt thus formed.

Non-limiting examples of pharmaceutically acceptable salts include but are not limited to: acetate, aspartate, benzenesulfonate, benzoate, bicarbonate, carbonate, halide (such as bromide, chloride, iodide, fluoride), bitartrate, citrate, salicylate, stearate, succinate, sulfate, tartrate, decanoate, edetate, fumarate, gluconate, and lactate or any combination thereof.

Additional examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Base addition salts can be prepared by 1) reacting the purified compound in its acid form with a suitable organic or inorganic base and 2) isolating the salt thus formed. Salts derived from appropriate bases include alkali metal (e.g., sodium, lithium, and potassium), alkaline earth metal (e.g., magnesium and calcium), ammonium and N+(C1-4alkyl)4 salts. This invention also envisions the quaternization of any basic nitrogen-containing groups of the compounds disclosed herein. Water or oil-soluble or dispersible products may be obtained by such quaternization.

Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

In some embodiments, the term “one or more” refers to any numerical value selected form of 1, 2, 3, 4, 5, or 6. In some embodiments, the heteroatom comprises any of N, O, NH, or S.

In some embodiments, the compounds described herein are chiral compounds (i.e. possess an asymmetric carbon atom). In some embodiments, diastereomers, geometric isomers and individual isomers are encompassed within the scope of the present invention. In some embodiments, a chiral compound described herein is in form of a racemic mixture. In some embodiments, a chiral compound is in form of a single enantiomer, with an asymmetric carbon atom having the R configuration. In some embodiments, a chiral compound is in form of a single enantiomer, with an asymmetric carbon atom having the S configuration as described hereinabove.

In some embodiments, a chiral compound is in form of a single enantiomer with enantiomeric purity of more than 70%. In some embodiments, a chiral compound is in form of a single enantiomer with enantiomeric purity of more than 80%. In some embodiments, a chiral compound is in form of a single enantiomer with enantiomeric purity of more than 90%. In some embodiments, a chiral compound is in form of a single enantiomer with enantiomeric purity of more than 95%.

In some embodiments, the compound of the invention comprising an unsaturated bond is in a form of a trans-, or cis-isomer. In some embodiments, the composition of the invention comprises a mixture of cis- and trans-isomers, as described hereinabove.

In some embodiments, the compounds described herein can exist in unsolvated form as well as in solvated form, including hydrated form. In general, the solvated form is equivalent to the unsolvated form and is encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solvate” refers to a complex of variable stoichiometry (e.g., di-, tri-, tetra-, penta-, hexa-, and so on), which is formed by a solute (the conjugate described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

Unless otherwise indicated, structures depicted herein are also meant to include all isomeric (e.g., enantiomeric, diastereomeric, geometric, conformational, and rotational) forms of the structure. For example, the R and S configurations for each asymmetric center, (Z) and (E) double bond isomers, and (Z) and (E) conformational isomers are included in this invention. As would be understood to one skilled in the art, a substituent can freely rotate around any rotatable bonds. Therefore, single stereochemical isomers as well as enantiomeric, diastereomeric, geometric, conformational, and rotational mixtures of the present compounds are within the scope of the invention.

Unless otherwise indicated, all tautomeric forms of the compounds of the invention are within the scope of the invention.

In some embodiments, the method further comprises inhibiting elastase secretion. In some embodiments, elastase secretion is from neutrophils. In some embodiments, the treating comprises inhibiting elastase secretion. In some embodiments, the method further comprises inhibiting elastase activity. In some embodiments, elastase activity comprises elastase secretion. In some embodiments, the treating comprises inhibiting elastase activity. In some embodiments, the agent inhibits elastase activity. In some embodiments, the agent inhibits elastase secretion. In some embodiments, the method further comprises administering an elastase secretion inhibitor. In some embodiments, an elastase secretion inhibitor is an elastase inhibitor. Elastase inhibitors are well known in the art and include for example, flavinoids, elafin, and various bacterial anti-elastase peptides. Any known elastase inhibitor may be used.

In some embodiments, the method does not comprise inhibiting elastase secretion. In some embodiments, the method does not comprise inhibiting elastase activity. In some embodiments, the treating does not comprise inhibiting elastase secretion. In some embodiments, the treating does not comprise inhibiting elastase activity. In some embodiments, the agent does not inhibit elastase secretion. In some embodiments, the agent does not inhibit elastase activity.

In some embodiments, the agent is linked to a nanoparticle. In some embodiments, the agent is inside a nanoparticle. In some embodiments, the agent is comprised within a nanoparticle. In some embodiments, the agent is encapsulated within a nanoparticle. In some embodiments, the agent is encapsulated by a nanoparticle. In some embodiments, the agent is linked to the surface of the nanoparticle. In some embodiments, the nanoparticle comprises the agent. In some embodiments, linked is covalently linked. In some embodiments, linked is non-specifically linked.

In some embodiments, the nanoparticle further comprises a neutrophil-targeting moiety. In some embodiments, the moiety is a peptide. In some embodiments, neutrophil-targeting moiety is linked to the surface of the nanoparticle. Examples of peptides that target to neutrophils and nanoparticles comprising same can be found in International Patent Application PCT/IL2021/050783, herein incorporated by reference in its entirety.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid. In some embodiments, a “polypeptide” and/or a “protein” comprises at least 50 amino acids. In some embodiments, a “peptide” comprises fewer than 50 amino acids. In some embodiments, a “polypeptide” and/or a “protein” comprises multiple peptide subunits.

The term “peptide” encompasses also the term “peptide analog”. The term “peptide analog” and “analog” are used herein interchangeably and refer to an analog of a peptide having at least 80% identity with the original peptide, wherein the analog retains the activity of the original peptide. Thus, the terms “analog” and “active analog” may be used interchangeably. The term “analog” refers to a peptide which contains substitutions, rearrangements, deletions, additions and/or chemical modifications in the amino acid sequence of the parent peptide. According to some embodiments, the peptide analog has at least 80%, at least 90% or at least 95% sequence identity to the original peptide. According to one embodiment, the analog has about 70% to about 95%, about 80% to about 90% or about 85% to about 95% sequence identity to the original peptide. According to some embodiments, the analog of the present invention comprises the sequence of the original peptide in which 1 or 2 deletions, additions and/or substitutions were made.

The term “peptide” encompasses also the term “peptide fragment”. The term “fragment” refers to a fragment of the original peptide or of an analog thereof in which 1 or 2 amino acid residues have been deleted, wherein said fragment retains the activity of the original peptide or analog. Thus, the terms “fragment” and “active fragment” may be used interchangeably.

The substitutions of the amino acids may be conservative or non-conservative substitution. The non-conservative substitution encompasses substitution of one amino acid by any other amino acid. In one particular embodiment, the amino acid is substituted by a non-natural amino acid.

The term “analog” encompasses also the term “conservative analog”. Conservative substitutions of amino acids as known to those skilled in the art are within the scope of the present invention. Conservative amino acid substitutions include replacement of one amino acid with another having the same type of functional group or side chain, e.g., aliphatic, aromatic, positively charged, negatively charged. One of skill will recognize that individual substitutions, refer to a “conservatively modified analog” where the alteration results in the substitution of an amino acid with a chemically similar amino acid. Conservative substitution tables providing functionally similar amino acids are well known in the art. One typical example of conservative substitution is provided below.

The following six groups each contain amino acids that are conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W). In other embodiments, the conservative substitution encompass substitution with a chemically similar non-natural amino acid.

Thus, in some embodiments, the analog is a conservative analog of the peptide. According to some embodiments, the conservative analog of the present invention comprises the sequence of the original peptide in which 1 or 2 conservative substitutions were made. According to another embodiment, the analog consists of the amino acid sequence of the original peptide in which 1 or 2 conservative substitutions were made. Thus, the analog consists of the amino acid sequence of the original peptide with 1 or 2 conservative substitutions.

The term “amino acid” as used herein refers to an organic compound comprising both amine and carboxylic acid functional groups, which may be either a natural or non-natural amino acid. The twenty-two natural amino acids are aspartic acid (Asp), tyrosine (Tyr), leucine (Leu), tryptophan (Trp), arginine (Arg), valine (Val), glutamic acid (Glu), methionine (Met), phenylalanine (Phe), serine (Ser), alanine (Ala), glutamine (Gln), glycine (Gly), proline (Pro), threonine (Thr), asparagine (Asn), lysine (Lys), histidine (His), isoleucine (Ile), cysteine (Cys), selenocysteine (Sec), and pyrrolysine (Pyl). Non-limiting examples of non-natural amino acids include diaminopropionic acid (Dap), diaminobutyric acid (Dab), ornithine (Orn), aminoadipic acid, β-alanine, 1-naphthylalanine, 3-(1-naphthyl)alanine, 3-(2-naphthyl)alanine, y-aminobutiric acid (GABA), 3-(aminomethyl) benzoic acid, p-ethynyl-phenylalanine, p-propargly-oxy-phenylalanine, m-ethynyl-phenylalanine, p-bromophenylalanine, p-iodophenylalanine, p-azidophenylalanine, p-acetylphenylalanine, azidonorleucine, 6-ethynyl-tryptophan, 5-ethynyl-tryptophan, 3-(6-chloroindolyl)alanine, 3-(6-bromoindolyl)alanine, 3-(5-bromoindolyl)alanine, azidohomoalanine, p-chlorophenylalanine, a-aminocaprylic acid, O-methyl-L-tyrosine, N-acetylgalactosamine-a-threonine, and N-acetylgalactosamine-a-serine. According to one embodiment, the substitution is substitution with a non-natural amino acid.

In some embodiments, a neutrophil-targeting peptide comprises an amino acid sequence selected from the group consisting of: KFPDLDSRRLPHMSL (SEQ ID NO: 1), LATTHMVFSPDH (SEQ ID NO: 2), PSSNLESTPLSLL (SEQ ID NO: 3), SSLMTTQLIATSI (SEQ ID NO: 4), PELDSKPYFPPL (SEQ ID NO: 5), ELVTASMPRPNN (SEQ ID NO: 6), SLESSPMAQLPQ (SEQ ID NO: 7), SELRSTPLLVPS (SEQ ID NO: 8), LQIQSWSSSP (SEQ ID NO: 9), STMTILGTGS (SEQ ID NO: 10), TETSLRIVSTNP (SEQ ID NO: 11), LSIVSGSALNHL (SEQ ID NO: 12), and LTLVSERPMI (SEQ ID NO: 13). In some embodiments, a human neutrophil-targeting peptide comprises a sequence selected from SEQ ID NO: 1-8. In some embodiments, a rodent neutrophil-targeting peptide comprises a sequence selected from SEQ ID NO: 9-13. In some embodiments, rodent is murine. In some embodiments, neutrophils are mature neutrophils.

In some embodiments, the agent is comprised in a composition of the invention. In some embodiments, contacting is contacting with a composition of the invention. In some embodiments, the administering is administering a composition of the invention.

By another aspect, there is provided a composition comprising a nanoparticle comprising an agent and a neutrophil-targeting moiety.

Agents that maybe used in the composition of the invention are provided hereinabove. In some embodiments, the agent is selected from a cyclin dependent kinase 4 (CDK4) inhibitor, a cyclin dependent kinase 6 (CDK6) inhibitor, an epidermal growth factor receptor (EGFR) inhibitor, a proto-oncogene tyrosine-protein kinase Src (SRC) inhibitor and a sodium (Na) channel blocker. In some embodiments, the agent is selected from a compound of Formula 1. In some embodiments, the agent is selected from Afatinib, Clomipramine, Nisoldiprine, Amitriptyline, Trifluoperazine, Abemaciclib, Ribociclib, Palbociclib Bosutinib, and Dyclonine. In some embodiments, the agent is selected from Afatinib, Clomipramine, Amitriptyline, Trifluoperazine, Abemaciclib, Ribociclib, Palbociclib Bosutinib, and Dyclonine. In some embodiments, the agent is Trifluoperazine.

In some embodiments, the neutrophil-targeting moiety is a neutrophil-targeting peptide. In some embodiments, the peptide comprises a sequence selected from SEQ ID NO: 1-13. In some embodiments, the peptide consists of a sequence selected from SEQ ID NO: 1-13.

According to some embodiments, the peptide comprises at most 500, 450, 400, 350, 300, 250, 200, 150, 100, 90, 80, 70, 60, 50, 45, 40, 35, 30, 25, 20, 15 or 10 amino acids. Each possibility represents a separate embodiment of the invention. In some embodiments, the peptide comprises at most 100 amino acids. In some embodiments, the peptide comprises at most 50 amino acids. In some embodiments, the peptide comprises at most 30 amino acids. In some embodiments, the peptide comprises up to 30 amino acids. In some embodiments, the peptide comprises at most 20 amino acids. In some embodiments, the peptide comprises at most 15 amino acids.

According to some embodiments, the peptide comprises 6-100, 6-50, 6-40, 6-30, 6-25, 6-20, 6-15, 5-12, 6-10, 10-100, 10-50, 10-40, 10-30, 10-25, 10-20, 10-15, 10-12, 12-100, 12-50, 12-40, 12-30, 12-25, 12-20, 12-15, 15-100, 15-50, 15-40, 15-30, 15-25, or 15-20, amino acids. Each possibility represents a separate embodiment of the present invention. According to some embodiments, the peptide comprises at least 6 amino acids. According to some embodiments, the peptide comprises at least 8 amino acids. According to some embodiments, the peptide comprises at least 10 amino acids. According to some embodiments, the peptide comprises at least 12 amino acids. According to some embodiments, the peptide comprises at least 15 amino acids.

According to some embodiments, the peptide comprises SEQ ID NO: 1. According to some embodiments, the peptide consists of SEQ ID NO: 1. According to some embodiments, the peptide comprises SEQ ID NO: 2. According to some embodiments, the peptide consists of SEQ ID NO: 2. According to some embodiments, the peptide comprises SEQ ID NO: 3. According to some embodiments, the peptide consists of SEQ ID NO: 3. According to some embodiments, the peptide comprises SEQ ID NO: 4. According to some embodiments, the peptide consists of SEQ ID NO: 4. According to some embodiments, the peptide comprises SEQ ID NO: 5. According to some embodiments, the peptide consists of SEQ ID NO: 5. According to some embodiments, the peptide comprises SEQ ID NO: 6. According to some embodiments, the peptide consists of SEQ ID NO: 6. According to some embodiments, the peptide comprises SEQ ID NO: 7. According to some embodiments, the peptide consists of SEQ ID NO: 7. According to some embodiments, the peptide comprises SEQ ID NO: 8. According to some embodiments, the peptide consists of SEQ ID NO: 8. According to some embodiments, the peptide comprises an amino acids sequence selected from the group consisting of SEQ ID NO: 1-8. According to some embodiments, the peptide consists of an amino acids sequence selected from the group consisting of SEQ ID NO: 1-8. In some embodiments, the peptide binds to human neutrophils and/or human CD177 and the peptide comprises or consists of an amino acids sequence selected from the group consisting of SEQ ID NO: 1-8.

According to some embodiments, the peptide comprises SEQ ID NO: 9. According to some embodiments, the peptide consists of SEQ ID NO: 9. According to some embodiments, the peptide comprises SEQ ID NO: 10. According to some embodiments, the peptide consists of SEQ ID NO: 10. According to some embodiments, the peptide comprises SEQ ID NO: 11. According to some embodiments, the peptide consists of SEQ ID NO: 11. According to some embodiments, the peptide comprises SEQ ID NO: 12. According to some embodiments, the peptide consists of SEQ ID NO: 12. According to some embodiments, the peptide comprises SEQ ID NO: 13. According to some embodiments, the peptide consists of SEQ ID NO: 13. According to some embodiments, the peptide comprises an amino acids sequence selected from the group consisting of SEQ ID NO: 9-13. According to some embodiments, the peptide consists of an amino acids sequence selected from the group consisting of SEQ ID NO: 9-13. In some embodiments, the peptide binds to human neutrophils and/or human CD177 and the peptide comprises or consists of an amino acids sequence selected from the group consisting of SEQ ID NO: 9-13.

According to some embodiments, the peptides comprise at least one cyclization. According to some embodiments, the peptides of the present invention are cyclic peptides. The term “cyclization” as used herein refers to an intramolecular bond between two non-adjacent amino acids. The terms “cyclic peptide” and “cyclopeptide” are used herein interchangeably and refer to a peptide having an intramolecular bond between two non-adjacent amino acids. The cyclization can be made through a covalent or non-covalent bond. Intramolecular bonds include, but are not limited to, backbone to backbone, sidechain to backbone and side-chain to side-chain bonds. According to some embodiments, the cyclization occurs between the N-terminal and C-terminal amino acids. According to some embodiments, the cyclization occurs via a spacer. According to some embodiments, the peptides are linear peptides.

According to some embodiments, the peptides of the present invention are conjugated to at least one moiety capable of increasing solubility. According to some embodiments, the peptides of the present invention are conjugated to at least one moiety capable of increasing permeability. According to some embodiments, the peptides of the present invention are conjugated at least one moiety capable of increasing solubility or permeability. According to some embodiments, at least one is a plurality of moieties. In some embodiments, a plurality is 2. In some embodiments, a plurality is at least 2, 3, 4, 5, 6, 7, 8, 9, or 10. Each possibility represents a separate embodiment of the invention.

According to other embodiments, the peptides of the present invention are conjugated to at least one linker or spacer. According to further embodiments, the peptides of the present invention are conjugated to at least one moiety capable of increasing solubility or permeability and optionally to at least one linker or spacer. According to yet further embodiments, the peptides of the present invention are conjugated to at least one moiety capable of increasing solubility or permeability and to at least one linker or spacer. According to yet further embodiments, the peptides of the present invention are conjugated to at least one moiety capable of increasing solubility or permeability and to at least one linker or spacer, wherein the at least one moiety capable of increasing solubility or permeability and the at least one linker or spacer are covalently linked to each other.

In some embodiments, the linker is an amino acid linker. In some embodiments, the linker is a chemical linker. In some embodiments, the linker is a bond. In some embodiments, the bond is a covalent bond. In some embodiments, the bond is a peptide bond. In some embodiments, the spacer is an amino acid spacer. In some embodiments, the linker or spacer comprises at least 1, 2, 3, 4, 5, 6, 7, 8, 9 or 10 amino acids. Each possibility represents a separate embodiment of the invention. In some embodiments, the linker or spacer is a single amino acid. In some embodiments, the linker or spacer comprises at most 1, 2, 3, 4, 5, 6, 7, 8, 9, 10, 11, 12, 13, 14, 15, 20, 25, 30, 35, 40, 45, 50, 60, 70, 75, 80, 90, or 100 amino acids. Each possibility represents a separate embodiment of the invention. In some embodiments, the linker or spacer comprises at most 1 amino acid. In some embodiments, the linker or spacer comprises at most 10 amino acids.

In some embodiments, the linker is a cysteine residue. In some embodiments, the linker is a lysine residue. In some embodiments, the linker is at least one repeat of the dipeptide lysine-alanine. In some embodiments, the linker is at least two repeats of the dipeptide KA. In some embodiments, the linker is two repeats of the dipeptide KA. In some embodiments, the linker comprises or consists of KAKA (SEQ ID NO: 16).

In some embodiments, a first peptide is separated from a second peptide by a spacer. In some embodiments, a first peptide is linked to a second peptide by a linker. In some embodiments, a peptide is separated from a moiety by a spacer. In some embodiments, a peptide is linked to a moiety by a linker. In some embodiments, a first moiety and a second moiety are separated by a spacer. In some embodiments, a first moiety and a second moiety are linked by a linker. In some embodiments, the linkage is a C-terminal linkage. In some embodiments, the linkage is an N-terminal linkage. In some embodiments, the linkage is not an N-terminal linkage. In some embodiments, there is no linkage to the N-terminus of the peptide. In some embodiments, the peptide comprises a free N-terminus.

According to some embodiments, the peptide is conjugated to at least one moiety via the peptide's C-terminus. According to some embodiments, the peptide is conjugated to the at least one linker or spacer via the peptide's C-terminus. According to some embodiments, the N-terminus of the peptide is not modified. According to other embodiments, the peptide has a free amine group on its N-terminus. Without being bound to any theory or mechanism, it is speculated that the amine group in the N-terminus of the peptides may be involved in the binding to neutrophils.

Moieties capable of increasing solubility are well known in the art and any such moiety may be employed for the peptide of the invention. Moieties that are capable of increasing solubility include but are not limited to: 8-amino-3,6-dioxaoctanoic acid (Doa) residues, polyethylene-glycol (PEG) in any length and peptides comprising the amino acid sequence GGGS (SEQ ID NO: 14) or GGGGS (SEQ ID NO: 15). In some embodiments, the moiety is a DOA residue. In some embodiments, the moiety is PEG. In some embodiments, the linker comprises at least one repeat of SEQ ID NO: 14. In some embodiments, the linker comprises at least one repeat of SEQ ID NO: 15. In some embodiments, the linker comprises or consists of at least 1, 2, 3, 4, or 5 repeats of SEQ ID NO: 14. Each possibility represents a separate embodiment of the invention. In some embodiments, the linker comprises or consists of at least 1, 2, 3, 4, or 5 repeats of SEQ ID NO: 15. Each possibility represents a separate embodiment of the invention.

According to specific embodiments, the moiety capable of increasing solubility comprises an 8-amino-3,6-dioxaoctanoic acid (Doa) residue. According to some embodiments, the peptides are conjugated to 1, 2, 3, 4 or 5 Doa residues. Each possibility represents a separate embodiment of the present invention. According to some embodiments, the peptides are conjugated to 2 Doa residues. According to further embodiments, the Doa residues are covalently connected to each other, to the peptide sequence and/or to a linker. According to some embodiments, the peptides are conjugated to two units of Doa residues covalently connected to each other. In some embodiments, the covalent linkage is a peptide linkage. In some embodiments, the peptide and the residue are in a single amino acid chain.

The terms “conjugated” or “peptide conjugate” as used herein refer to a molecule in which a peptide moiety is attached (i.e., coupled or linked), either directly or via a linker or spacer, by means of covalent chemical bonding to at least one peptidic or non-peptidic molecule. In some embodiments, the nanoparticle is decorated with the peptide. In some embodiments, decorated is conjugated.

The terms “linker” and “spacer” are used herein interchangeably and refer to any molecule that covalently binds and therefore linking two molecules. Non-limiting examples of the linker are amino acids, peptides, or any other organic substance that can be used to allow distance between two linked molecules. According to specific embodiments, the linker is a flexible linker. According to specific embodiments, the linker is a flexible peptide. According to further specific embodiments, the linker is a flexible peptide comprising at least one glycine residue. According to particular embodiments, the linker comprises plurality of Lysine residues. According to some specific embodiments, the linker comprises 3-12 Lysine residues. According to particular embodiments, the linker comprises a 3-maleimidopropionic acid (Mpa) residue.

According to some embodiments, the present invention provides peptide conjugates comprising at least one peptide selected from SEQ ID NO: 1 to 13 and at least one moiety capable of increasing solubility. According to other embodiments, the present invention provides peptide conjugates comprising at least one peptide selected from SEQ ID NO: 1 to 13 and at least one linker or spacer. According to further embodiments, the peptide conjugates comprise at least one peptide selected from SEQ ID NO: 1 to 13, at least one moiety capable of increasing solubility and optionally at least one linker or spacer. According to yet further embodiments, the peptide conjugates comprise at least one peptide selected from SEQ ID NO: 1 to 13, at least one moiety capable of increasing solubility and at least one linker or spacer. According to yet further embodiments, the peptide conjugates comprise at least one moiety capable of increasing solubility and at least one linker or spacer, wherein the at least one moiety capable of increasing solubility and the at least one linker or spacer are covalently linked to each other.

According to some embodiments, the peptide conjugates comprise at least one peptide selected from SEQ ID NO: 1 to 13 and 1, 2, 3, 4 or 5 units of a Doa residue. Each possibility represents a separate embodiment of the present invention. According to further embodiments, the peptide conjugates comprise at least one peptide selected from SEQ ID NO: 1 to 13 and 2, 3, 4 or 5 units of a Doa residue, wherein the Doa residues are covalently connected to each other to the peptide sequence and/or to a linker.

According to further embodiments, the peptide conjugates comprise at least one peptide selected from SEQ ID NO: 1 to 13, at least one Doa residue and at least one Mpa residue.

According to some embodiments, the peptide conjugate comprises at least one moiety capable of increasing solubility, wherein the at least one moiety is conjugated to the peptide via the peptide's C-terminus. According to some embodiments, peptide conjugate comprises at least one linker or spacer, wherein the at least one linker or spacer is conjugated to the peptide via the peptide's C-terminus. According to some embodiments, the N-terminus of the peptide conjugate is not modified. According to other embodiments, the peptide conjugate has a free amine group on its N-terminus.

According to some embodiments, the peptide conjugate has a structure according to Formula III:

Peptide-Doa-Doa-C (Formula III)

wherein “C” is a Cysteine residue, and wherein “Peptide” denotes a peptide of the invention. In some embodiments, “Peptide” denotes a peptide of the invention.

In some embodiments, a peptide multimer, comprising a plurality of is conjugated to the nanoparticle. According to some embodiments, the plurality of peptides is a plurality of same peptide. According to some embodiments, the plurality of peptides is a plurality of different peptides. According to some embodiments, the peptides are identical or different peptides. According to some embodiments, the present invention provides a peptide multimer comprising a plurality of identical or different peptides selected from peptides of the invention. According to other embodiments, the present invention provides a peptide multimer for use in targeting to human neutrophils, wherein the peptide multimer comprises a plurality of identical or different peptides selected from the group consisting of SEQ ID NO: 1-8. According to other embodiments, the present invention provides a peptide multimer for use in targeting to murine neutrophils, wherein the peptide multimer comprises a plurality of identical or different peptides selected from the group consisting of SEQ ID NO: 9-13.

The terms “peptide multimer” and “multimeric peptide” are used interchangeably herein and refer to a construct that contains a plurality (at least two, typically at least three or more) of peptides, not necessarily adjacent.

According to some embodiments, the peptide multimer is a branched molecule. According to other embodiments, the peptide multimer is a non-branched molecule. According to other embodiments, the peptide multimer is a linear molecule. According to other embodiments, the peptide multimer is a circular molecule.

According to some embodiments, the peptide multimer comprises at most 4, 6, 8, 10, 12, 14, 16, 18, 20, 25, 30, 35, 40, 45 or 50 peptides. Each possibility represents a separate embodiment of the present invention. According to some embodiments, the peptide multimer comprises at least 2, 4, 6, 8, 10, 12, 14, or 16 peptides. Each possibility represents a separate embodiment of the present invention. According to some embodiments, the peptide multimer comprises 2-20, 2-16, 2-14, 2-12, 2-10, 2-8, 2-6 or 2-4 identical or different peptides. Each possibility represents a separate embodiment of the present invention. According to some embodiments, the peptide multimer comprises 2-20, 2-16, 2-14, 2-12, 2-10, 2-8, 2-6 or 2-4 peptides. Each possibility represents a separate embodiment of the present invention. According to some embodiments, the peptide multimer comprises 2-20 peptides. According to some embodiments, the peptide multimer comprises 2-4 peptides. According to some embodiments, the peptide multimer comprises 2 peptides. According to some embodiments, the peptide multimer comprises 4 peptides. According to some embodiments, the peptide multimer comprises 16 peptides. According to specific embodiments, the peptide multimer comprises 4 identical or different peptides.

According to some embodiments, the peptides in the peptide multimer are covalently linked to each other directly or through a linker or spacer. According to other embodiments, the peptides in the peptide multimer are covalently linked to a scaffold directly or through a linker or spacer.

According to some embodiments, the peptide conjugates in the peptide multimer are covalently linked to each other directly or through a linker or spacer. According to other embodiments, the peptide conjugates in the peptide multimer are covalently linked to a scaffold directly or through a linker or spacer. According to further embodiments, the peptide conjugates in the peptide multimer are non-covalently linked to a scaffold directly or through a linker or spacer. According to some embodiments, the scaffold is a branched scaffold. According to other embodiments, the scaffold is a non-branched scaffold.

According to some embodiments, each one of the peptides or peptide conjugates is bound to the scaffold directly or via a linker or spacer. According to other embodiments, the peptides or peptide conjugates are covalently attached to each other and at least one peptide/peptide conjugate is bound to the scaffold directly or via a linker or spacer.

According to some embodiments, the scaffold is a peptidic or polypeptidic scaffold. According to other embodiments, the peptidic or polypeptidic scaffold connects the peptides to each other on a single location in the scaffold, or to a different location on a scaffold. Each possibility represents a separate embodiment of the invention. According to some embodiments, the scaffold comprises at least one Lysine (Lys) residue. According to other embodiments, the scaffold comprises at least three Lys residues. According to further embodiments, the at least three Lys residues are connected together by amide bonds to form a branched multimeric scaffold. According to some embodiments, at least one amide bond is formed between the epsilon amine of a Lys residue and the carboxy group of another Lys residue.

According to a particular embodiment, the peptide multimer comprises the molecule Mpa-Cysteine-peptide.

According to a particular embodiment, the peptide multimer comprises a molecule of the scheme:

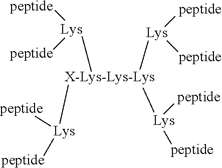

wherein X represents the peptide's C-terminus selected from carboxy acid, amide or alcohol group and optionally a linker or spacer, and each “peptide” independently denotes a peptide of the invention.

According to some specific embodiments, at least one of the peptides is present in multiple copies. According to some embodiments, the multiple copies are linked thereby forming a multi-target peptide multimer. According to some embodiments, the peptide copies are linked through a linker. According to other embodiments, the peptide copies are linked directly. According to further embodiments, the multimer comprises copies linked both directly and via a linker.

According to some embodiments, the peptide multimer comprises a plurality of neutrophil-binding peptides arranged in an alternating sequential polymeric structure B(X₁X₂X₃ . . . Xₘ)ₙB or in a block copolymer structure B(X₁)ₙZ(X₂)ₙZ(X₃)ₙZ . . . (Xₘ)ₙ, wherein B is an optional sequence of 1-10 amino acid residues; n is at each occurrence independently an integer of 2-50; m is an integer of 3-50; each of X₁, X₂ . . . Xₘ is an identical or different peptide of the invention; Z at each occurrence is a bond or a spacer of 1-4 amino acid residues. Each possibility represents a separate embodiment of the present invention.

The term “block copolymer structure” means that all the copies of a single peptide contained in the multimer are arranged adjacently.

According to some embodiments, the scaffold comprises or formed from a polyethylene glycol (PEG) molecule(s) or a modified PEG molecule(s). According to certain embodiments, the scaffold comprises a branched PEG molecule. According to some embodiments, the branched molecule comprises at least two sites available to bind a peptide of the present invention. According to other embodiments, the scaffold comprises from 2 to 100, 3 to 90, 4 to 60, 5 to 50, 6 to 40, 7 to 35, 8 to 30, 9 to 25 or 10 to 20, or 2 to 50 sites available to bind a peptide.

According to some embodiments, the PEG molecule is a branched molecule, comprising at least two separate connections to a peptide. According to other embodiments, the PEG is bound to additional PEG molecules. According to certain embodiments, multiple PEG molecules are bound to provide a multi-armed PEG molecule. According to certain embodiments, the peptides are connected to the PEG scaffold through amide bonds formed between amino groups of an NH₂—PEG molecule. According to yet other embodiments, at least one peptide is connected to PEG scaffold though a Lys residue.

According to some embodiments, the peptide multimer comprises a branched scaffold comprising at least one Lys residue linked to the peptides or peptide conjugates directly or through a spacer or linker. According to specific embodiments, the peptide multimer comprises a branched scaffold comprising at two Lys residues linked to the peptides or peptide conjugates directly or through a spacer or linker. According to further specific embodiments, the peptide multimer comprises a branched scaffold comprising the amino acid sequence Lys-Ala-Lys-Ala (KAKA, SEQ ID NO: 16) linked to the peptides or peptide conjugates directly or through a spacer or linker.

According to some embodiments, the peptide multimer further comprises a biotin moiety covalently attached to said peptide multimer directly or via a spacer or linker. According to some embodiments, the biotin is attached to said peptide multimer through the C-terminus. The biotin moiety makes the peptide multimer accessible for fluorescent detection and manipulation. According to other embodiments, the peptide multimer further comprises a biotin moiety, wherein the biotin moiety is non-covalently attached to said peptide multimer. According to some embodiments, the peptide multimer further comprises an avidin moiety attached to said peptide multimer directly or via a spacer or linker. According to some embodiments, the peptide multimer further comprises a streptavidin moiety attached to said peptide multimer directly or via a spacer or linker. According to further embodiments, the peptide multimer comprises a biotin moiety and an avidin/streptavidin moiety attached to each other through biotin-avidin interactions.

According to some embodiments, the multimeric peptide is homo-multimeric. According to other embodiments, the multimeric peptide is hetero-multimeric.

As used herein, the term “homo-multimeric” refers to a multimeric peptide comprising multiple copies of a single peptide. According to some embodiments, the multimeric peptide comprises 2-20, 2-16, 2-14, 2-12, 2-10, 2-8, 2-6 or 2-4 identical peptides. According to some embodiments, the multimeric peptide comprises 2-20, 2-16, 2-14, 2-12, 2-10, 2-8, 2-6 or 2-4 copies of a peptide comprising the amino acid sequence KFPDLDSRRLPHMSL (SEQ ID NO: 1). Each possibility represents a separate embodiment of the invention. According to some embodiments, the multimeric peptide comprises 2-20, 2-16, 2-14, 2-12, 2-10, 2-8, 2-6 or 2-4 copies of a peptide comprising the amino acid sequence LATTHMVFSPDH (SEQ ID NO: 2). Each possibility represents a separate embodiment of the invention. According to some embodiments, the multimeric peptide comprises 2-20, 2-16, 2-14, 2-12, 2-10, 2-8, 2-6 or 2-4 copies of a peptide comprising the amino acid sequence PSSNLESTPLSLL (SEQ ID NO: 3). Each possibility represents a separate embodiment of the invention. According to some embodiments, the multimeric peptide comprises 2-20, 2-16, 2-14, 2-12, 2-10, 2-8, 2-6 or 2-4 copies of a peptide comprising the amino acid sequence SSLMTTQLIATSI (SEQ ID NO: 4). Each possibility represents a separate embodiment of the invention. According to some embodiments, the multimeric peptide comprises 2-20, 2-16, 2-14, 2-12, 2-10, 2-8, 2-6 or 2-4 copies of a peptide comprising the amino acid sequence PELDSKPYFPPL (SEQ ID NO: 5). Each possibility represents a separate embodiment of the invention. According to some embodiments, the multimeric peptide comprises 2-20, 2-16, 2-14, 2-12, 2-10, 2-8, 2-6 or 2-4 copies of a peptide comprising the amino acid sequence ELVTASMPRPNN (SEQ ID NO: 6). Each possibility represents a separate embodiment of the invention. According to some embodiments, the multimeric peptide comprises 2-20, 2-16, 2-14, 2-12, 2-10, 2-8, 2-6 or 2-4 copies of a peptide comprising the amino acid sequence SLESSPMAQLPQ (SEQ ID NO: 7). Each possibility represents a separate embodiment of the invention. According to some embodiments, the multimeric peptide comprises 2-20, 2-16, 2-14, 2-12, 2-10, 2-8, 2-6 or 2-4 copies of a peptide comprising the amino acid sequence SELRSTPLLVPS (SEQ ID NO: 8). Each possibility represents a separate embodiment of the invention. According to some embodiments, the multimeric peptide comprises 2-20, 2-16, 2-14, 2-12, 2-10, 2-8, 2-6 or 2-4 copies of a peptide comprising the amino acid sequence LQIQSWSSSP (SEQ ID NO: 9). Each possibility represents a separate embodiment of the invention. According to some embodiments, the multimeric peptide comprises 2-20, 2-16, 2-14, 2-12, 2-10, 2-8, 2-6 or 2-4 copies of a peptide comprising the amino acid sequence STMTTLGTGS (SEQ ID NO: 10). Each possibility represents a separate embodiment of the invention. According to some embodiments, the multimeric peptide comprises 2-20, 2-16, 2-14, 2-12, 2-10, 2-8, 2-6 or 2-4 copies of a peptide comprising the amino acid sequence TETSLRIVSTNP (SEQ ID NO: 11). Each possibility represents a separate embodiment of the invention. According to some embodiments, the multimeric peptide comprises 2-20, 2-16, 2-14, 2-12, 2-10, 2-8, 2-6 or 2-4 copies of a peptide comprising the amino acid sequence LSIVSGSALNHL (SEQ ID NO: 12). Each possibility represents a separate embodiment of the invention. According to some embodiments, the multimeric peptide comprises 2-20, 2-16, 2-14, 2-12, 2-10, 2-8, 2-6 or 2-4 copies of a peptide comprising the amino acid sequence LTLVSERPMI (SEQ ID NO: 13). Each possibility represents a separate embodiment of the invention. According to specific embodiments, the multimeric peptide is a tetramer comprising 4 copies of a peptide comprising an amino acid sequence selected from the group consisting of SEQ ID NO: 1 to 13. Each possibility represents a separate embodiment of the present invention. According to specific embodiments, the multimeric peptide is a tetramer comprising 4 copies of a peptide comprising the amino acid sequence selected from SEQ ID NO: 1 to 8. According to specific embodiments, the multimeric peptide is a tetramer comprising 4 copies of a peptide comprising the amino acid sequence selected from SEQ ID NO: 9 to 13. According to other embodiments, the multimeric peptide is a tetramer comprising 4 copies of a peptide comprising the amino acid sequence KFPDLDSRRLPHMSL (SEQ ID NO: 1). According to other embodiments, the multimeric peptide is a tetramer comprising 4 copies of a peptide comprising the amino acid sequence LQIQSWSSSP (SEQ ID NO: 9).

The term “hetero-multimeric” as used herein refers to a multimeric peptide comprising one or more copies of at least two different peptides. The term “different peptides” refers to peptides having different sequence and not to two copies of the same peptide. According to some embodiments, the multimeric peptide comprises one or more copies of at least two different peptides of the invention. According to specific embodiments, the multimeric peptide comprises one or more copies of at least two different peptides of the invention.

According to some embodiments, the hetero multimeric peptide comprises 2, 3, 4, 5, 6, 7 or 8 different peptide sequences of the invention.

It has been shown that a peptide multimer comprising 4 copies of a single neutrophil-binding peptide, binds more efficiently to circulating neutrophils than a monomer of the peptide. Without being bound to any theory or mechanism, it is believed that a hetero-multimeric peptide comprising at least two substantially different peptides would target even higher percent of neutrophils than a homo-multimeric peptide.

According to some embodiments, the hetero-multimeric peptide comprises 2-20, 2-10 or 2-5 copies of at least one of the different peptides. According to other embodiments, the hetero-multimeric peptide comprises 2-20, 2-10 or 2-5 copies of at least two of the different peptides. According to further embodiments, the hetero-multimeric peptide comprises 2-20, 2-10 or 2-5 copies of at least three of the different peptides. Each possibility represents a separate embodiment of the present invention.

According to specific embodiments, the peptide multimer is a tetramer peptide presenting the neutrophil-binding peptide on 4 branches. According to further embodiments, at least one peptide in the tetramer has a free amine group on its N-terminus. According to yet further embodiments, each one of the neutrophil-binding peptides in the tetramer has a free amine group on the N-terminus. According to specific embodiments, the multimer peptide is a tetramer peptide presenting 4 copies of one neutrophil-binding peptide on 4 branches. According to further specific embodiments, the multimer peptide is a tetramer peptide presenting 4 copies of one neutrophil-binding peptide on 4 branches, wherein at least one copy of the neutrophil-binding peptide has a free amine group on its N-terminus. According to further embodiments, the multimer peptide is a tetramer peptide presenting 4 copies of one neutrophil-binding peptide on 4 branches, wherein each one of the copies of the neutrophil-binding peptide has a free amine group on its N-terminus.

According to some specific embodiments, the peptide multimer comprises a structure according to Formula I.

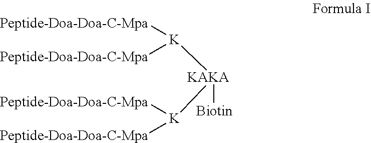

wherein each “Peptide” independently denotes a peptide of the invention.

According to some embodiments, the peptide multimer comprises a structure according to Formula I, wherein each “Peptide” independently denotes a peptide of the invention, wherein at least 2 peptides are different. According to other embodiments, the peptide multimer comprises a structure according to Formula I, wherein each “Peptide” independently denotes a peptide of the invention, wherein at least 3 peptides are different. According to other embodiments, the peptide multimer comprises a structure according to Formula I, wherein each “Peptide” independently denotes a different peptide sequence, wherein each peptide sequence comprises a sequence selected from the group consisting of SEQ ID NO: 1 to 13. According to some embodiments, the peptide multimer comprises a structure according to Formula I, wherein each “Peptide” independently denotes a peptide of the invention, wherein at least 2 peptides are identical. According to other embodiments, the peptide multimer comprises a structure according to Formula I, wherein each “Peptide” independently denotes a peptide of the invention, wherein at least 3 peptides are identical. According to yet other embodiments, the peptide multimer comprises a structure according to Formula I, wherein “Peptide” denotes a peptide of the invention. According to other embodiments, the peptide multimer comprises a structure according to Formula I, wherein each “Peptide” independently denotes a peptide of the invention, wherein 4 peptides are identical.

According to some embodiments, the peptide multimer comprises a structure according to Formula I, wherein “Peptide” denotes a peptide comprising the amino acid sequence of SEQ ID NO: 1. According to some embodiments, the peptide multimer comprises a structure according to Formula I, wherein “Peptide” denotes a peptide consisting of the amino acid sequence of SEQ ID NO: 1. According to some embodiments, the peptide multimer comprises a structure according to Formula I, wherein “Peptide” denotes a peptide comprising the amino acid sequence of SEQ ID NO: 9. According to some embodiments, the peptide multimer comprises a structure according to Formula I, wherein “Peptide” denotes a peptide consisting of the amino acid sequence of SEQ ID NO: 9.

According to some specific embodiments, the peptide multimer comprises a structure according to Formula II:

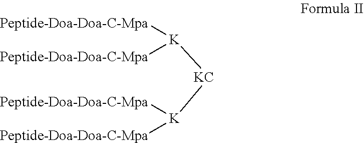

wherein each “Peptide” independently denotes a peptide of the invention

According to some embodiments, the peptide multimer comprises a structure according to Formula II, wherein each “Peptide” independently denotes a peptide of the invention, wherein at least 2 peptides are different. According to other embodiments, the peptide multimer comprises a structure according to Formula II, wherein each “Peptide” independently denotes a peptide of the invention, wherein at least 3 peptides are different. According to other embodiments, the peptide multimer comprises a structure according to Formula II, wherein each “Peptide” independently denotes a different peptide sequence, wherein each peptide sequence comprises a sequence selected from the group consisting of SEQ ID NO: 1 to 13. According to some embodiments, the peptide multimer comprises a structure according to Formula II, wherein each “Peptide” independently denotes a peptide of the invention, wherein at least 2 peptides are identical. According to other embodiments, the peptide multimer comprises a structure according to Formula II, wherein each “Peptide” independently denotes a peptide of the invention, wherein at least 3 peptides are identical. According to yet other embodiments, the peptide multimer comprises a structure according to Formula II, wherein “Peptide” denotes a peptide of the invention. According to other embodiments, the peptide multimer comprises a structure according to Formula II, wherein each “Peptide” independently denotes a peptide of the invention, wherein 4 peptides are identical.

According to some embodiments, the peptide multimer comprises a structure according to Formula II, wherein “Peptide” denotes a peptide comprising the amino acid sequence of SEQ ID NO: 1. According to some embodiments, the peptide multimer comprises a structure according to Formula II, wherein “Peptide” denotes a peptide consisting of the amino acid sequence of SEQ ID NO: 1. According to some embodiments, the peptide multimer comprises a structure according to Formula II, wherein “Peptide” denotes a peptide comprising the amino acid sequence of SEQ ID NO: 9. According to some embodiments, the peptide multimer comprises a structure according to Formula II, wherein “Peptide” denotes a peptide consisting of the amino acid sequence of SEQ ID NO: 9.

According to another aspect, there is provided a peptide complex comprising at least two peptide multimers of the invention.

According to some embodiments, the present invention provides a peptide complex comprising at least two peptide multimers. As used herein, the term “peptide complex” refers to a construct that contains a plurality (at least two, typically at least three or more) of identical or different peptide multimers, not necessarily adjacent. According to some embodiments, the peptide complex comprises at least two peptide multimers, wherein the peptide multimers comprise at least one peptide of the invention. According to certain embodiments, the present invention provides a peptide complex for use in targeting to human neutrophils wherein the peptide complex comprises at least two peptide multimers, wherein the peptide multimers comprise at least one peptide comprising a sequence selected from SEQ ID NO: 1 to 13. According to some embodiments, the peptide complex comprises at least two peptide multimers, wherein the peptide multimers comprise at least two peptides comprising the amino acid sequence of SEQ ID NO: 9. According to some embodiments, the peptide complex comprises at least two peptide multimers, wherein the peptide multimers comprise at least two peptides comprising the amino acid sequence of SEQ ID NO: 1. According to certain embodiments, the present invention provides a peptide complex for use in targeting to murine neutrophils wherein the peptide complex comprises at least two peptide multimers, wherein the peptide multimers comprise at least one peptide comprising SEQ ID NO: 9. According to certain embodiments, the present invention provides a peptide complex for use in targeting to human neutrophils wherein the peptide complex comprises at least two peptide multimers, wherein the peptide multimers comprise at least one peptide comprising SEQ ID NO: 1. According to some embodiments, the peptide multimers in the peptide complex are covalently connected to each other directly or via a linker or spacer. According to some embodiments, the linker or spacer is selected from the group consisting of, but not limited to, amino acids, peptides, and any other organic substance that can be used to allow distance between two linked molecules. According to other embodiments, the peptide multimers in the peptide complex are non-covalently attached to each other. According to some embodiments, the peptide multimers in the peptide complex are non-covalently attached to each other through a biotin-avidin interactions. According to some embodiments, the peptide complex comprises at least two biotin moieties and an avidin/streptavidin moiety, wherein the at least two biotin moieties are covalently attached to the peptide multimers, and wherein the avidin/streptavidin moiety is non-covalently attached to the biotin moieties. According to specific embodiments, the peptide complex comprises 4 peptide multimers and an avidin/streptavidin moiety, wherein each one of the peptide multimers is covalently attached to a biotin moiety, and wherein the 4 peptide multimers are non-covalently attached to the avidin/streptavidin moiety.

In the context of the present invention, the term “nanoparticle” refers to a particle having an average size of up to about 1000 nm, as determined by any method known in the art, for example dynamic light scattering (DLS) for determining the hydrodynamic diameter of the particles and transmission electron microscopy (TEM) for determining the accurate geometric nanoparticle size. According to some embodiments, the size of the nanoparticle is within the range of 50-1000, 100-1000, 200-1000, 250-1000, 300-1000, 500-1000, 600-1000, 700-1000, 50-900, 100-900, 200-900, 250-900, 300-900, 500-900, 600-900, 700-900, 50-800, 100-800, 200-800, 250-800, 300-800, 500-800, 600-800, 700-800, 50-600, 100-600, 200-600, 250-600, 300-600, 500-600, or 50-200 nm. Each possibility represents a separate embodiment of the present invention. According to some embodiments, the nanoparticle is selected from a nanosphere and a nanorod. As used herein, the term “nanosphere” refers to a nanoparticle having a spherical shape. The term “nanorod” refers to a nanoparticle having a rod-like shape. According to some embodiments, the nanoparticle is a liposome. According to other embodiments, the nanoparticle is a polymeric nanoparticle. According to additional embodiments, the polymeric nanoparticle comprises poly(lactic-co-glycolic acid) (PLGA). According to additional embodiments, the nanoparticle is a metallic nanoparticle. According to particular embodiments, the nanoparticle is fluorescently-labeled. According to some embodiments, the nanoparticle is modified with PEG. According to some embodiments, the nanoparticle comprises a PLGA core. In some embodiments, the nanoparticle is coated with a reactive agent. In some embodiments, the reactive agent is suitable for conjugating the peptide, peptide multimer or peptide complex to the nanoparticle. Reactive groups for conjugation are well known in the art and examples of such are provided herein below. According to some embodiments, the reactive group is a binding or capture group. In some embodiments, the binding or capture group is for binding or capturing a peptide or a linker or spacer. In some embodiments, the reactive group is streptavidin (SA). In some embodiments, the SA is for binding a peptide/multimer/complex comprising biotin. In some embodiments, the reactive group is a thiol. In some embodiments, the thiol if for binding a peptide/multimer/complex comprising a cysteine. In some embodiments, the cysteine is a free cystine. In some embodiments, the cysteine is in a linker. In some embodiments, the linkage is a maleimide linkage.

In some embodiments, the nanoparticle encapsulates the agent. In some embodiments, the nanoparticle comprises a hydrophilic core and the agent is in the core. In some embodiments, the nanoparticle comprises a hydrophobic core and the agent is in the core. In some embodiments, the agent is associated with the surface of the nanoparticle. In some embodiments, the agent is linked or conjugated to the surface of the nanoparticle. In some embodiments, the agent is a hydrophobic agent. In some embodiments, the agent is a hydrophilic agent.

In some embodiments, the nanoparticle is a core particle with the peptide and agent adhered thereto. According to some embodiments, the core particle is a streptavidin-coated nanoparticle and the peptide, peptide multimer or complex comprises at least one biotin moiety, wherein the peptide, peptide multimer or complex is attached to the core particle through biotin-streptavidin interactions. According to specific embodiment, streptavidin-coated particle is a streptavidin-coated fluorescent nile red particle.

According to other embodiments, the core particle is a polymeric nanoparticle wherein the polymeric nanoparticle is selected from the group consisting but not limited to: polylactide-polyglycolide copolymers (PLGA), polylactide, polycaprolactones, polyacrylates, polyethylene glycol (PEG) and propylene glycol (PPG) nanoparticles and copolymers thereof.

According to specific embodiments, the polymeric nanoparticle is a PLGA nanoparticle. According to particular embodiments, the PLGA is acid-terminated. According to further embodiments, the PLGA particle further comprises PEG. In some embodiments, the PLGA is PEG modified PLGA. In some embodiments, the PLGA particle further comprises Maleimide. In some embodiments, the particle is a PLGA-PEG-Maleimide particle. In some embodiments, the Maleimide is 30% Maleimide.

According to some embodiments, the core particle is a streptavidin-coated PLGA nanoparticle, and the at least one peptide or peptide multimer/complex comprises at least one biotin moiety, wherein the plurality of the at least one peptide or peptide multimer/complex are attached to the outer surface of said core particle through biotin-streptavidin interactions.

According to other embodiments, the core particle is a PLGA particle comprising acid-terminated PLGA, and the at least one peptide or peptide multimer/complex comprises at least one peptide sequence with a free amine group on the C-terminus, wherein the plurality of the at least one peptide or peptide multimer/complex are directly conjugated to the outer surface of said core particle through covalent amide bonds.

According to some embodiments, the nanoparticle is coated with a plurality of peptide conjugates. According to some embodiments, the nanoparticle is coated with a plurality of peptide conjugates having a structure according to Formula III. According to some embodiments, the nanoparticle comprises a PLGA core coated with a plurality of peptide conjugates. According to some embodiments, the nanoparticle comprises a PLGA core coated with a plurality of peptide conjugates having a structure according to Formula III.

The agent can be either encapsulated inside the nanoparticle (e.g., encapsulation in liposomes or polymeric nanoparticles) or attached to the particle surface non-covalently or covalently via a spacer or linker.

In some embodiments, the agent is a therapeutic agent. In some embodiments, the composition is a therapeutic composition. In some embodiments, the composition is a pharmaceutical composition. In some embodiments, the composition is formulated for systemic administration. In some embodiments, the composition is formulated for administration to a subject. In some embodiments, the composition is formulated for administration to the lungs. In some embodiments, the composition is formulated for inhalation. In some embodiments, the composition is formulated for intravenous administration.

According to some embodiments, the composition further comprises a pharmaceutically acceptable carrier, excipient or adjuvant. The term “pharmaceutically acceptable carrier” or “pharmaceutically acceptable excipient” or “pharmaceutically acceptable adjuvant” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. Non-limiting examples of suitable excipients are water, saline, phosphate buffered saline (PBS), dextrose, glycerol, ethanol, or the like and combinations thereof. Other suitable carriers are well known to those skilled in the art. The use of such media and agents for pharmaceutically active substances is well known in the art.

The compositions may contain other active compounds providing supplemental, additional, or enhanced therapeutic functions. The peptides or peptide multimers/complexes of the present invention could be, according to some embodiments, suspended in a sterile saline solution for therapeutic uses. Numerous suitable drug delivery systems are known and include, e.g., implantable drug release systems, hydrogels, hydroxymethylcellulose, microcapsules, liposomes, microemulsions, microspheres, and the like. Controlled release preparations can be prepared through the use of polymers to complex or adsorb the molecule according to the present invention. For example, biocompatible polymers include matrices of poly(ethylene-co-vinyl acetate) and matrices of a polyanhydride copolymer of a stearic acid dimer and sebacic acid. The rate of release of the molecule according to the present invention from such a matrix depends upon the molecular weight of the molecule, the amount of the molecule within the matrix, and the size of dispersed particles.

The carrier may comprise, in total, from about 0.1% to about 99.99999% by weight of the pharmaceutical compositions presented herein.

It is well known in the art that neutrophil activation while required for innate immune response can become pathologic. This can occur due to overactivation, aberrant activation, and the like. It often occurs after an infection/disease has stimulated the innate immune system, and then the system continues to attach healthy cells and tissue. The method therefore relates to treating neutrophil mediated pathology such as neutrophil-mediated inflammation and neutrophil-mediated tissue damage.

In some embodiments, the composition is for use in a method of the invention. In some embodiments, the method is for treating neutrophil-mediated inflammation. In some embodiments, the method is for treating a neutrophil-associated disease or condition. In some embodiments, the treating comprises treating neutrophil-mediated inflammation. In some embodiments, the treating comprises reducing neutrophil-mediated inflammation. In some embodiments, the treating comprises reducing ROS production by neutrophils. In some embodiments, neutrophil-mediated inflammation is pathological neutrophil-mediated inflammation. In some embodiments, treating is treating neutrophil-mediated tissue damage. In some embodiments, neutrophil-mediated inflammation comprises neutrophil-mediated tissue damage.

In some embodiments, treating comprises reduction of a side effect of the agent. In some embodiments, the side effect is a side effect of the free agent. In some embodiments, a free agent is an agent not in a nanoparticle comprising a neutrophil targeting peptide. In some embodiments, the nanoparticle is a lipid-based nanoparticle. In some embodiments, the lipid-based nanoparticle is a liposome. In some embodiments, treating with a neutrophil targeting peptide composition comprising an agent reduced a side effect of the agent. In some embodiments, the side effect is elevated liver enzyme levels. In some embodiments, elevated is elevated in blood. In some embodiments, elevated in elevated in a tissue. In some embodiments, the liver enzyme is selected from alanine aminotransferase (ALT), aspartate transaminase (AST), alkaline phosphatase (ALP) and gamma-glutamyl transpeptidase (GGT). In some embodiments, the liver enzyme is ALT. In some embodiments, a side effect is inhibition of neutrophil differentiation. In some embodiments, differentiation is differentiation in the bone marrow. In some embodiments, a side effect is neutropenia.

In some embodiments, the treating is treating a disease or condition characterized by neutrophil-mediated inflammation. In some embodiments, the treating is treating a disease or condition that comprises neutrophil-mediated inflammation. In some embodiments, the inflammation is mediated by ROS. In some embodiments, the inflammation is caused by ROS. In some embodiments, the tissue damage is mediated by ROS. In some embodiments, the tissue damage comprises elevated liver enzyme levels. In some embodiments, the tissue damage is caused by ROS. In some embodiments, ROS is ROS production. In some embodiments, the ROS is ROS produced by neutrophils. In some embodiments, the ROS production is production by neutrophils.

In some embodiments, the tissue is a target tissue. In some embodiments, the tissue is a damaged tissue. In some embodiments, the tissue is an inflamed tissue. In some embodiments, the tissue is lung. In some embodiments the tissue is the colon. In some embodiments, the tissue is liver. In some embodiments, the neutrophils are lung neutrophils. In some embodiments, the neutrophils are liver neutrophils.

In some embodiments, the treating is treating prooncogenic neutrophils. In some embodiments, the neutrophils are tumor associated neutrophils (TANs). In some embodiments, the treating is inhibiting prooncogenic neutrophil function. In some embodiments, the prooncogenic neutrophil function is ROS production. In some embodiments, the neutrophil-associated disease is cancer. In some embodiments, the cancer is a solid cancer. In some embodiments, the cancer is a hematological cancer. In some embodiments, the cancer is selected from breast cancer, lung cancer, colon cancer, pancreatic cancer, liver cancer, head and neck cancer and kidney cancer. In some embodiments, the cancer is breast cancer. In some embodiments, the cancer is lung cancer. In some embodiments, the cancer is liver cancer. It has been shown that ROS production by TANs can have a protumor effect as it can impair T cell function against the tumor. In such cases, the specific reduction of ROS is beneficial. Further, inhibition of ROS function while maintaining other anticancer neutrophil functions (such as NETs, granulation, etc.) is greatly beneficial. In some embodiments, the cancer is characterized by the presence of TANs. In some embodiments, the TANs are myeloid derived suppressor cells (MDSCs). In some embodiments, the cancer is characterized by the presence of ROS. In some embodiments, the ROS is in the tumor microenvironment.

In some embodiments, the method is for treating a subject. In some embodiments, the subject suffers from a disease or condition. In some embodiments, the subject suffers from a disease or condition that comprises or is mediated by neutrophil-mediated inflammation. In some embodiments, the subject is a subject in need of the treatment. In some embodiments, the subject is a mammal. In some embodiments, the subject is a human. In some embodiments, the subject is a rodent. In some embodiments, the rodent is a mouse.

In some embodiments, the disease or condition is selected from an inflammatory disease, an autoimmune disease, cancer, acute tissue damage and an infectious disease. In some embodiments, the disease is selected from an inflammatory disease, an autoimmune disease, cancer and an infectious disease. In some embodiments, the disease is selected from an inflammatory disease, an autoimmune disease and an infectious disease. In some embodiments, the condition is selected from an inflammatory condition, an autoimmune condition, cancer and an infectious condition. In some embodiments, the condition is selected from an inflammatory condition, an autoimmune condition and an infectious condition. In some embodiments, the disease or condition is an inflammatory disease or condition. In some embodiments, the disease or condition is lung inflammation. In some embodiments, the disease or condition is liver inflammation. In some embodiments, the disease or condition is an autoimmune disease or condition. In some embodiments, the disease or condition is cancer. In some embodiments, the disease or condition is an infectious disease or condition. In some embodiments, the disease or condition is a disease or condition characterized by neutrophil-mediated pathology. In some embodiments, the disease or condition comprises neutrophil mediated pathology. In some embodiments, the pathology is inflammation. In some embodiments, the pathology is tissue damage. In some embodiments, the tissue is lung. In some embodiments, the tissue is liver. In some embodiments, the disease or condition is a lung disease or condition. In some embodiments, the disease or condition is a liver disease or condition. In some embodiments, the tissue is a digestive tissue. In some embodiments, the disease or condition is a digestive tissue disease or condition. In some embodiments, the digestive tissue is bowel, intestine, or colon. In some embodiments, the pathology is immune suppression. In some embodiments, the immune suppression is T cell suppression. It has been shown that ROS can reduce the expression of the T cell receptor zeta chain and thus impair T cell activation; target T cells for necrotic-like cell death; inhibit NF-kB activation in T cells and induce CD8 T cell tolerance. Further, ROS can increase the genotoxicity (mutational load) in the tumor.

In some embodiments, the disease or condition is selected from acute respiratory distress syndrome (ARDS), chronic obstructive pulmonary disease (COPD), inflammatory bowel disease (IBD), sepsis, liver disease, bacterial pneumonia, and viral pneumonia. In some embodiments, the disease or condition is selected from acute respiratory distress syndrome (ARDS), chronic obstructive pulmonary disease (COPD), inflammatory bowel disease (IBD), sepsis, bacterial pneumonia, and viral pneumonia. In some embodiments, the disease or condition is ARDS. In some embodiments, the disease or condition is COPD. In some embodiments, the disease or condition is acute lung injury (ALI). In some embodiments, the disease or condition is emphysema. Diseases and conditions of lung inflammation are well known in the art and include for example, COPD, ARDS, emphysema, ALI, asthma, bronchiectasis, cystic fibrosis, Idiopathic pulmonary fibrosis (IPF) and infection. In some embodiments, lung inflammation comprises a disease or condition selected from COPD, ARDS, ALI and emphysema. In some embodiments, lung inflammation comprises a disease or condition selected from COPD, ARDS, ALI, infection and emphysema. In some embodiments, lung inflammation comprises a disease or condition selected from COPD and ARDS. In some embodiments, lung inflammation comprises lung infection. In some embodiments, lung infection is respiratory infection. In some embodiments, the disease or condition is IBD. In some embodiments, IBD comprises colitis and Crohn's disease. In some embodiments, the disease is colitis. In some embodiments, colitis is ulcerative colitis. In some embodiments, the disease is Crohn's disease. In some embodiments, the disease or condition is sepsis. In some embodiments, the disease or condition is liver disease. In some embodiments, the liver disease comprises liver fibrosis. In some embodiments, the liver disease is liver inflammation. In some embodiments, the liver disease is hepatitis. In some embodiments, the liver disease is cirrhosis. In some embodiments, the liver disease is acute liver injury. In some embodiments, the liver disease is chronic liver injury. In some embodiments, the liver disease is non-alcoholic fatty liver disease. In some embodiments, the liver disease is liver ischemia. In some embodiments, the liver disease is liver cancer. In some embodiments, the liver cancer is hepatocellular carcinoma. In some embodiments, the disease or condition is infection. In some embodiments, the infection is a bacterial infection. In some embodiments, the infection is a viral infection. In some embodiments, the infection is pneumonia. In some embodiments, the pneumonia is viral pneumonia. In some embodiments, the pneumonia is bacterial pneumonia. In some embodiments, the viral infection is a coronavirus infection. In some embodiments, the coronavirus is SARS-Cov-2. According to some embodiments, the autoimmune disease, condition or disorder is an inflammatory autoimmune disease, condition or disorder. Other examples of infections include peritonitis. Other examples of autoimmune diseases include Alzheimer disease.

In some embodiments, the disease is neutrophil-mediated skin disease. According to some embodiments, the inflammatory disease is a disease in which neutrophils are involved in the pathogenesis. According to some embodiments, the inflammatory disease or disorder is selected from the group consisting of peritonitis, colitis, vasculitis, atherosclerosis, chronic obstructive pulmonary disease (COPD), acute respiratory distress syndrome (ARDS), bronchiectasis, neutrophilic asthma, rheumatoid arthritis (RA), lupus, cystic fibrosis (CF), sepsis, multiple sclerosis, psoriasis and traumatic injury. Each possibility represents a separate embodiment of the present invention. According to some embodiments, the disease or disorder is Systemic Lupus Erythrocytes (SLE). According to some embodiments, the disease or disorder is rheumatoid arthritis (RA).

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intramuscular, or intraperitoneal.

According to some embodiments, the pharmaceutical composition is administered by an invasive mode of administration such as intramuscularly, intravenously, intra-arterially, intraarticularly or parenterally. According to specific embodiments, the pharmaceutical composition is administered intravenously. According to some embodiments, the composition is administered systemically. According to some embodiments, the composition is administered to a site of inflammation. According to some embodiments, the composition is formulated for systemic administration. According to some embodiments, the composition is formulated for administration to a site of inflammation.

The dosage administered will be dependent upon the age, health, and weight of the recipient, kind of concurrent treatment, if any, frequency of treatment, and the nature of the effect desired.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As used herein, the term “alkyl” describes an aliphatic hydrocarbon including straight chain and branched chain groups and usually comprising between 1 and 30, or between 1 and 10 carbon atoms. The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (bonded through a ring carbon) or heterocyclyl (bonded through a ring carbon) as defined herein. or “carboxylate”

The term “carbonyl” describes a —C(O)R′ group, where R′ is as defined hereinabove. The above-terms also encompass thio-derivatives thereof (thiocarboxy and thiocarbonyl).

The term “thiocarbonyl” describes a —C(S)R′ group, where R′ is as defined hereinabove. A “thiocarboxy” group describes a —C(S)OR′ group, where R′ is as defined herein. A “sulfinyl” group describes an —S(O)R′ group, where R′ is as defined herein. A “sulfonyl” or “sulfonate” group describes an —S(O)2R′ group, where R′ is as defined herein.

A “carbamyl” or “carbamate” group describes an —OC(O)NR′R″ group, where R′ is as defined herein and R″ is as defined for R′. A “nitro” group refers to a —NO2 group. The term “amide” as used herein encompasses C-amide and N-amide. The term “C-amide” describes a —C(O)NR′R″ end group or a —C(O)NR′-linking group, as these phrases are defined hereinabove, where R′ and R″ are as defined herein. The term “N-amide” describes a —NR″C(O)R′ end group or a —NR′C(O)— linking group, as these phrases are defined hereinabove, where R′ and R″ are as defined herein.

A “cyano” or “nitrile” group refers to a —CN group. The term “azo” or “diazo” describes an —N═NR′ end group or an —N═N— linking group, as these phrases are defined hereinabove, with R′ as defined hereinabove. The term “guanidine” describes a —R′NC(N)NR″R′″ end group or a —R′NC(N)NR″— linking group, as these phrases are defined hereinabove, where R′, R″ and R′″ are as defined herein. As used herein, the term “azide” refers to a —N3 group. The term “sulfonamide” refers to a —S(O)2NR′R″ group, with R′ and R″ as defined herein.

The term “phosphonyl” or “phosphonate” describes an —OP(O)—(OR′)2 group, with R′ as defined hereinabove. The term “phosphinyl” describes a —PR′R″ group, with R′ and R″ as defined hereinabove. The term “alkylaryl” describes an alkyl, as defined herein, which substituted by an aryl, as described herein. An exemplary alkylaryl is benzyl.

The term “heteroaryl” describes a monocyclic or fused ring (i.e. rings which share an adjacent pair of atoms) group having in the ring(s) one or more atoms, such as, for example, nitrogen, oxygen and sulfur and, in addition, having a completely conjugated pi-electron system. As used herein, the term “heteroaryl” refers to an aromatic ring in which at least one atom forming the aromatic ring is a heteroatom. Heteroaryl rings can be foamed by three, four, five, six, seven, eight, nine and more than nine atoms. Heteroaryl groups can be optionally substituted. Examples of heteroaryl groups include, but are not limited to, aromatic C₃₋₈ heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2(1H)-quinolinonyl, 4-oxo-1,4-dihydroquinolinyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H)quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydro-benzoxazolyl, 1,4-naphthoquinolyl, 5,6,7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo-[3,4-b]indolyl, 1H-2,3,4,5-tetrahydro-azepino[3,4-b]indolyl, 1H-2,3,4,5-tetrahydroazepino-[4,3-b]indolyl, 1H-2,3,4,5-tetrahydro-azepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]pryidyl, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl. In some embodiments, heteroaryl groups are optionally substituted. In one embodiment, the one or more substituents are each independently selected from among halo, hydroxy, amino, cyano, nitro, alkylamido, acyl, C₁₋₆-alkyl, C₁₋₆-haloalkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl.

Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, O—C₁₋₆-alkyl, C₁₋₆-alkyl, hydroxy-C₁₋₆-alkyl and amino-C₁₋₆-alkyl.

As used herein, the terms “halo” and “halide”, which are referred to herein interchangeably, describe an atom of a halogen, that is fluorine, chlorine, bromine or iodine, also referred to herein as fluoride, chloride, bromide and iodide.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Human Blood Samples:

Human experiments were done based on Hadassah's Helsinki approval.

Following blood withdrawal, blood was overlaid on 3% dextran in 0.9% NaCl in a 1:1 ratio and left at RT for 25 min to allow erythrocyte sedimentation. Residual RBCs were lysed by adding water for 30 sec and stopped by adding 2.5% BSA/5×PBS. WBCs were then diluted in 0.5% BSA/1×PBS and total number of cells was counted. Purification of pure neutrophils (HDNs) was done by overlaying blood after RBC sedimentation in a 1:1 ratio on Histopaque-1077. After 30 min centrifugation at 400 g, pure neutrophils were found at the bottom of the tube (pellet). Mononuclear cells (LDF—low density fraction) settle between the Histopaque-1077 and the 0.9% saline.

### Example 1: Screen for ROS Inhibiting Drugs

A high-throughput screen was performed to find molecules that inhibit ROS production by neutrophils. Extracted human neutrophils were purified and kept in culture. The cells were then exposed to various drugs and ROS production was monitored over time. Compounds that also scavenged hydrogen peroxide (H₂O₂) and/or lowered neutrophil ATP levels (reflecting toxicity) were removed. The top hits from the screen were selected for further analysis.

160 basal hits were tested for their ability to inhibit neutrophil ROS production. 64 molecules were able to inhibit ROS production in the presence of PMA activation. 67 molecules were able to inhibit ROS production in the presence of N-Formylmethionine-leucyl-phenylalanine (fMLP). Of those 47 molecules were able to inhibit ROS production by both stimulation methods.

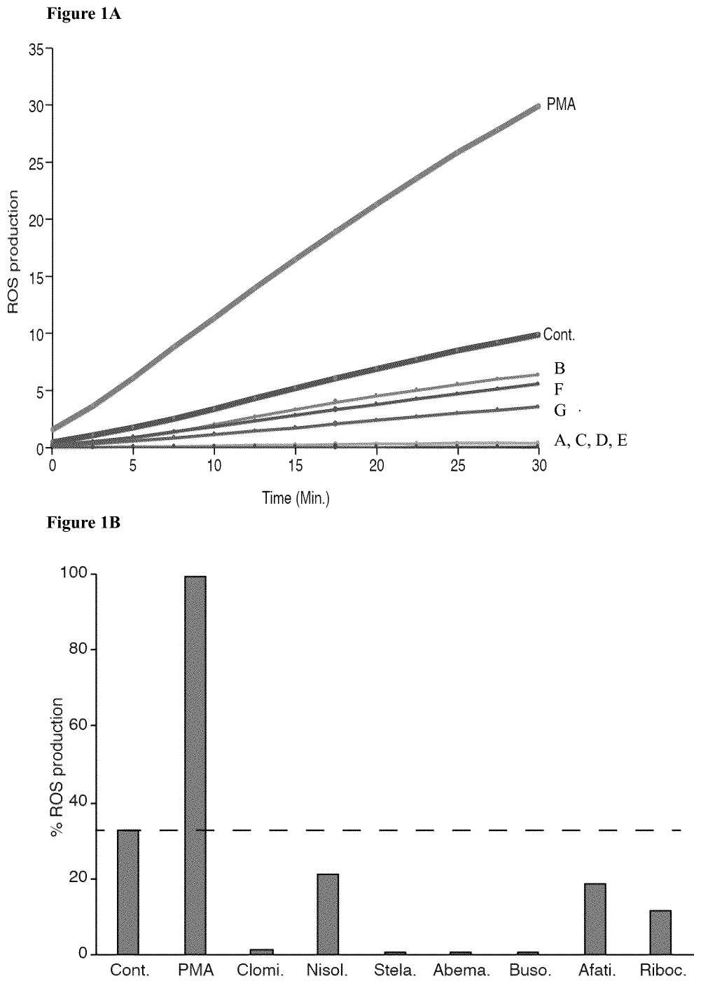

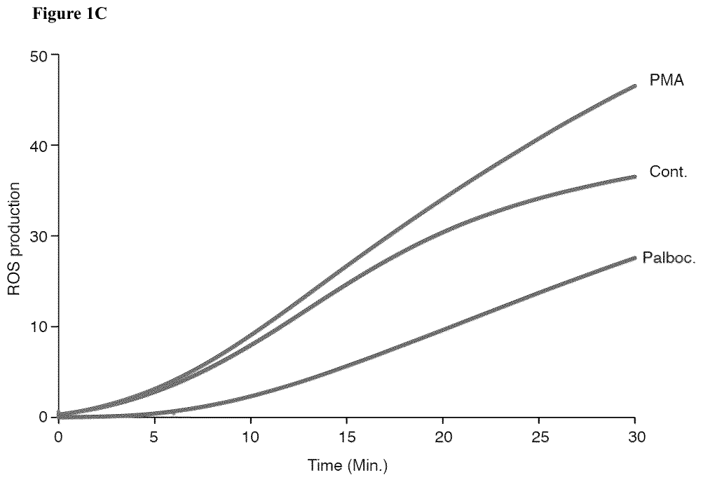

The effects of 7 FDA approved molecules that were highly ranked in the screen were examined further. Neutrophils not exposed to PMA were used as a negative control and neutrophils exposed to only PMA were used as a positive control. All seven tested molecules, as predicted, reduced ROS production in the presence of PMA (FIG. 1A-B, 10 uM concentration). Indeed, all seven were able to reduce ROS levels to below that of the control cells not treated with PMA. Interestingly, three of the molecules (molecules B, F and G, corresponding to Nisoldipine, Afatinib and Ribociclib) produced a more modest reduction, while the other four molecules (molecules A, C, D and E, corresponding to Clomipramine, Stelazine, Abemaciclib, and Bosutinib) were found to be more effective (FIG. 1B). These molecules could essentially completely block ROS production at this concentration.

Molecules D and G were both found to be CDK4/6 inhibitors (Abemaciclib and Ribociclib, respectively). While molecule E was a SRC inhibitor (Bosutinib) and molecules C and F were EGFR inhibitors (Stelazine and Afatinib, respectively). Molecules A and B are both tricyclic antidepressants (TCAs, Clomipramine and Nisoldiprine, respectively) which are known to be sodium (Na+) channel blockers. Stelazine (molecule C) is also a known Na channel blocker, and a third TCA, Amitriptyline, was also present in the 47 molecules from the screen that inhibited ROS production. Both SRC and EGFR are known in some cell systems to be upstream to CDK4/6 activation, although it is not yet known if their inhibition produces an effect on ROS via CDK4/6 or via a parallel pathway.

TCAs are so called due to their characteristic three ring structure (FIG. 6H-I). Stelazine shares this same structure (FIG. 6F), though it is not an antidepressant but rather an antipsychotic. 14 other tricyclic molecules (FIG. 6J) were also found in the neutrophil ROS inhibition screen, indicating that this structure is common to molecules that inhibit ROS from activated neutrophils.

In order to further confirm the universality of the effect of CDK4/6 inhibition on ROS production, a third CDK4/6 inhibitor, Palbociclib, was also tested. This CDK4/6 inhibitor also strongly reduced ROS production in the presence of PMA (FIG. 1C), even to below the levels observed from unstimulated neutrophils.

Finally, a ninth compound, another Na channel blocker (Dyclonine), was tested. At 10 μM, this compound also reduced ROS levels to below those measured from unstimulated neutrophils (44% of negative control cells).

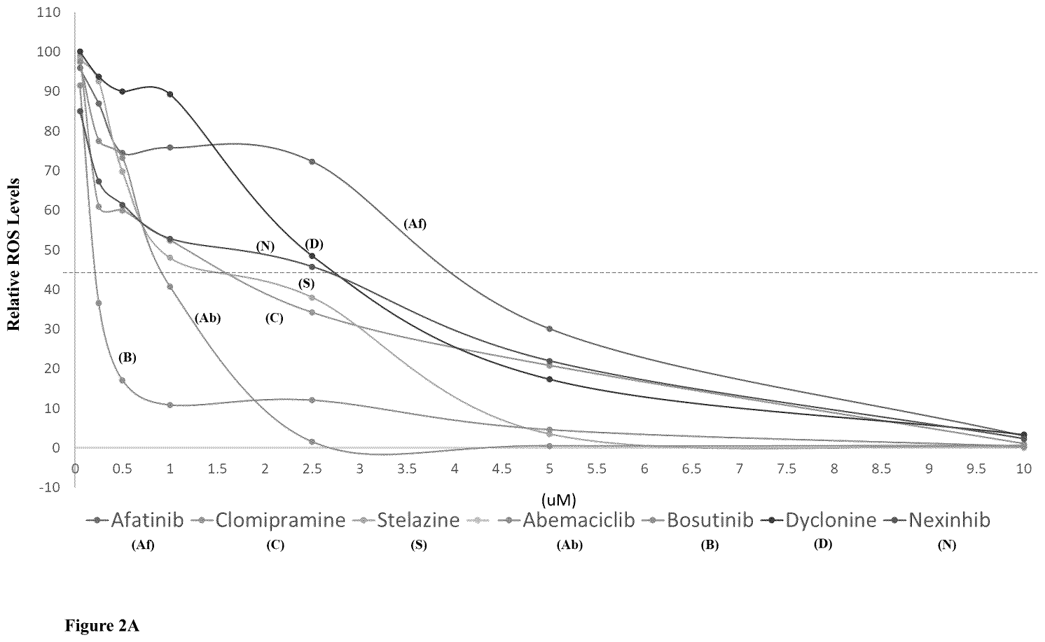

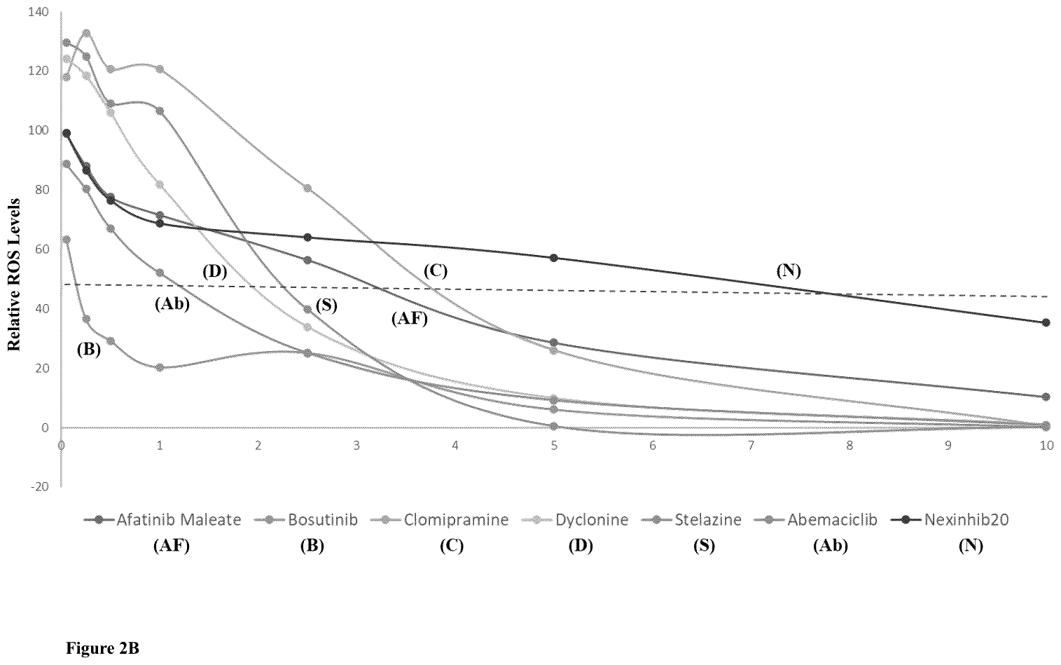

Bosutinib, Stelazine, Clomipramine, Abemaciclib, Afatanib and Dyclonine were selected for further characterization. As many of the molecules showed complete inhibition at 10 uM, various concentrations from 100 nm to 10 uM were tested. PMA was again used to stimulate ROS production in neutrophils and the effects of the drugs were monitored at increasing concentration levels. Nexinhib, a known exocytosis inhibitor, is known to also inhibit ROS and so was used as a positive control. The results from one experiment using humanneutrophils are presented in FIG. 2A. These experiments were repeated several times and the average results are presented in Table 1. Bosutnib, Abemaciclib, Stelazine and Clomipramine were found to be the most effective ROS inhibitors, though all of the tested molecules were at least as effective as Nexinhib. The molecules were all also effective in mouse neutrophils although to a generally lesser extent (FIG. 2B and Table 2). Once again FIG. 2B shows a single experiment, while the data presented in Table 2 is an average of several experiments.

TABLE 1
ROS inhibition in human neutrophils

| | | |
|---|---|---|
| Compound | ROS Inhibition IC50 | <90% ROS Inhibition |

| | | | | |
|---|---|---|---|---|
| Bosutinib | 200 | nM | 750 | nM |
| Abemaciclib | 1 | uM | 2 | uM |
| Stelazine | 1 | uM | 4 | uM |
| Clomipramine | 1 | uM | 5 | uM |
| Nexinhib | 1.5 | uM | 5 | uM |
| Dyclonine | 2.5 | uM | 7 | uM |
| Afatanib | 4 | uM | 9 | uM |

TABLE 2
ROS inhibition in mouse neutrophils

| Compound | ROS Inhibition IC50 | | <90% ROS Inhibition | |
|---|---|---|---|---|
| Bosutinib | 150 | nM | 1.5 | uM |
| Abemacicib | 1.5 | uM | 5 | uM |
| Dyclonine | 2 | uM | 5 | uM |
| Stelazine | 2.5 | uM | 4 | uM |
| Afatanib Maleate | 2.5 | uM | 8 | uM |
| Clomipramine | 3.5 | uM | 7 | uM |

| | | | |
|---|---|---|---|
| Nexinhib | 8 | uM | — |

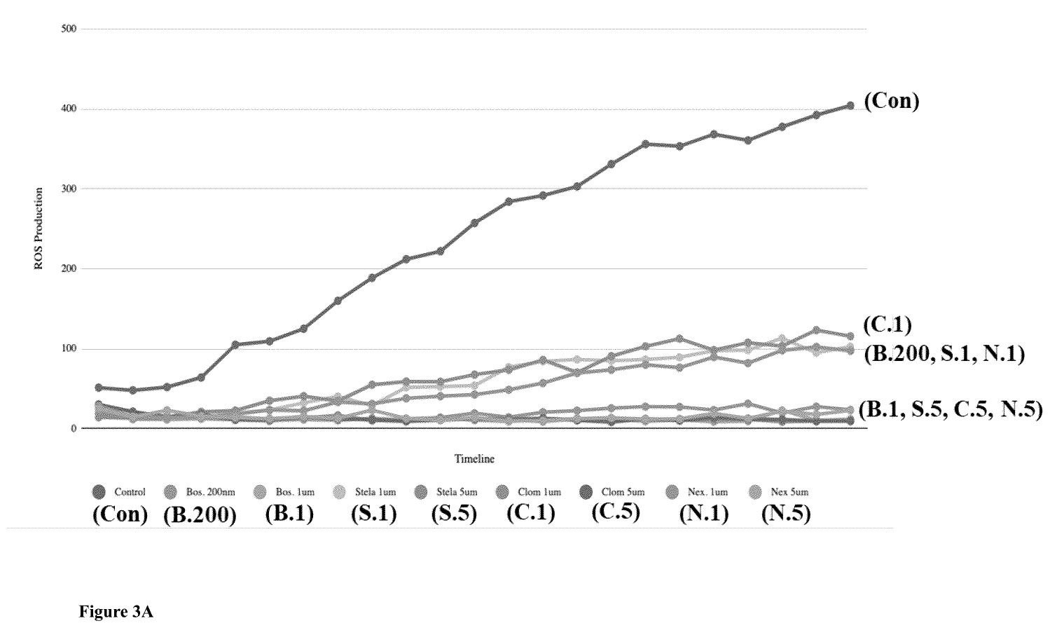

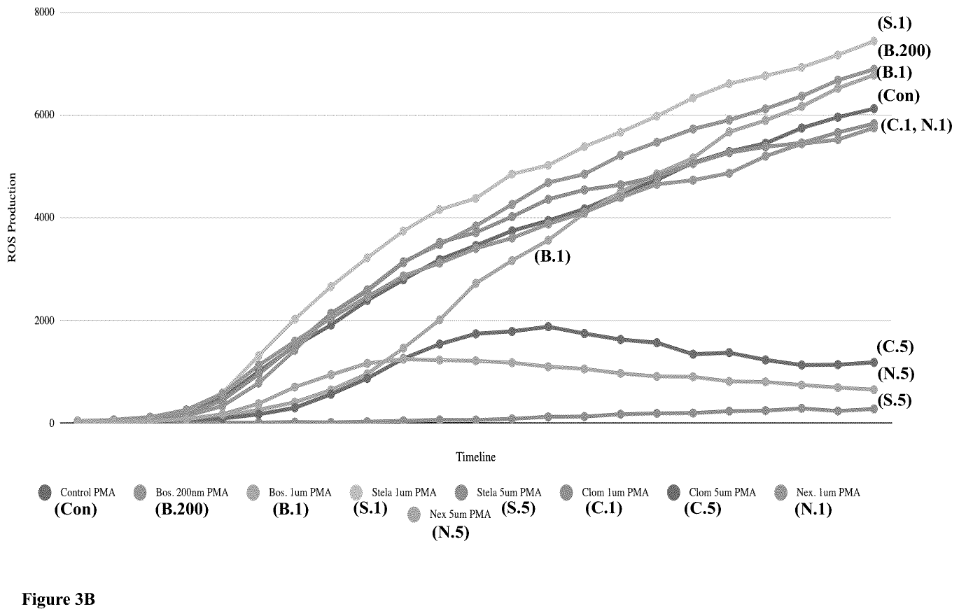

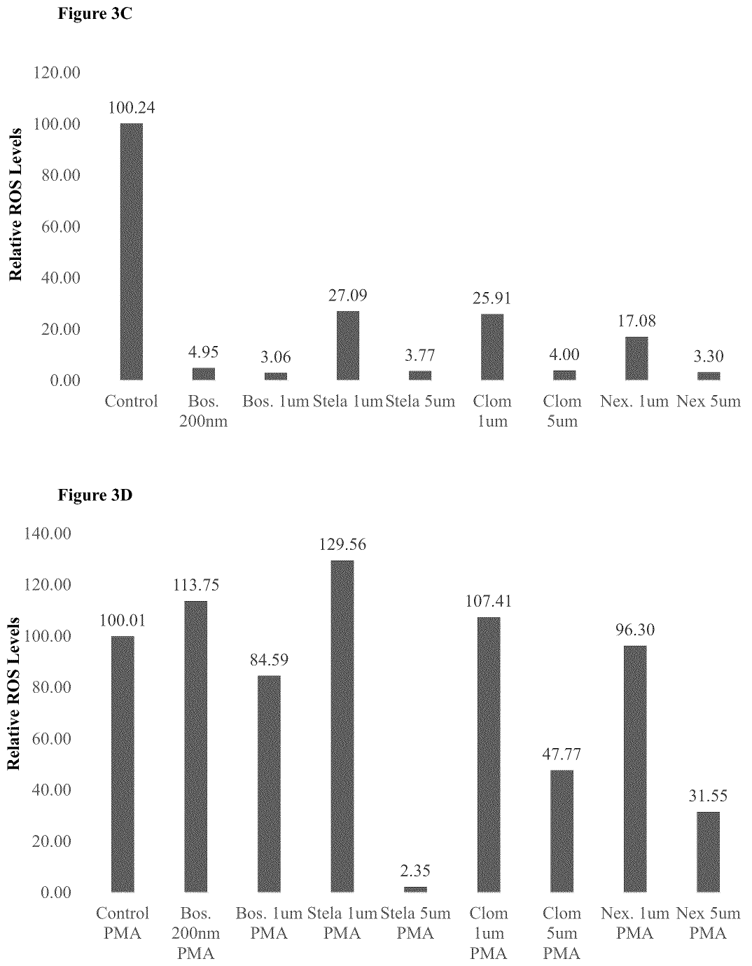

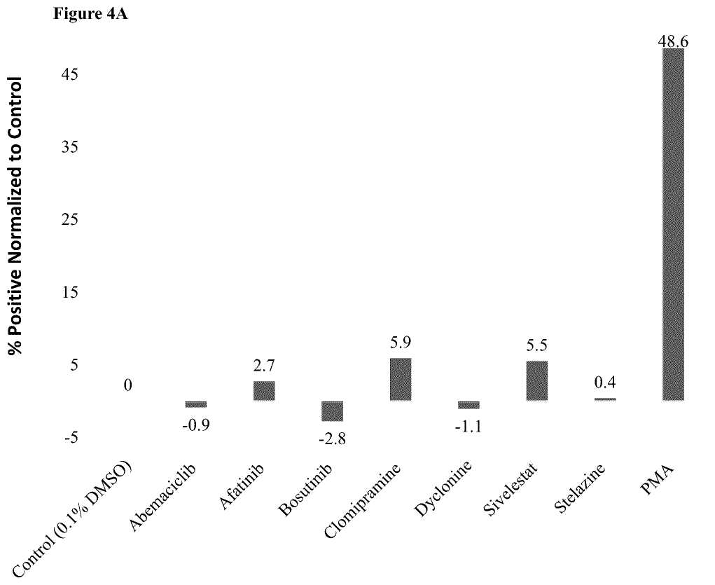

As all of the molecules were found effective when administered to isolated neutrophils, the most promising molecules (Bosutinib, Stelazine, Clomipramine and Nexinhib) were next tested on whole blood samples from healthy human subjects. A high and low concentration were tested for each compound. First, the compounds were tested on untreated whole blood samples, and were all found to reduce ROS levels (FIG. 3A). Not surprisingly the high concentrations produced more robust inhibition (FIG. 3C). Next, the compounds were tested on whole blood treated with PMA. After PMA stimulation the lower doses were all found to be ineffective in lowering ROS levels, but the higher doses of Stelazine, Clomipramine and Nexinhib were all highly effective producing greater than a 50% reduction in ROS (FIG. 3B). Indeed, the 5 uM Stelazine was by far the most effective producing a greater than 90% reduction in ROS, while 5 uM Clomipramine and Nexinhib produced a less strong effect (FIG. 3D). Because Bosutinib had been so effective in the tests on purified neutrophils, 200 nM was used as the low concentration and 1 uM as the high. Although initially the 1 uM dose produced ROS inhibition similar to 5 uM Clomipramine and Nexinhib this inhibition was quickly lost, and ROS levels reached those of controls. A 5 uM dose of Bosutinib is highly effective at ROS inhibition similar to the other 4 drugs.

### Example 2: Effect on Neutrophil Survival

The effect of the various drugs on neutrophil survival was measured. It is reported that some of the tested drugs may cause neutropenia. It was hypothesized that this is likely due to inhibition of neutrophil differentiation in the bone marrow and not due to actual neutrophil toxicity in fully differentiated neutrophils.

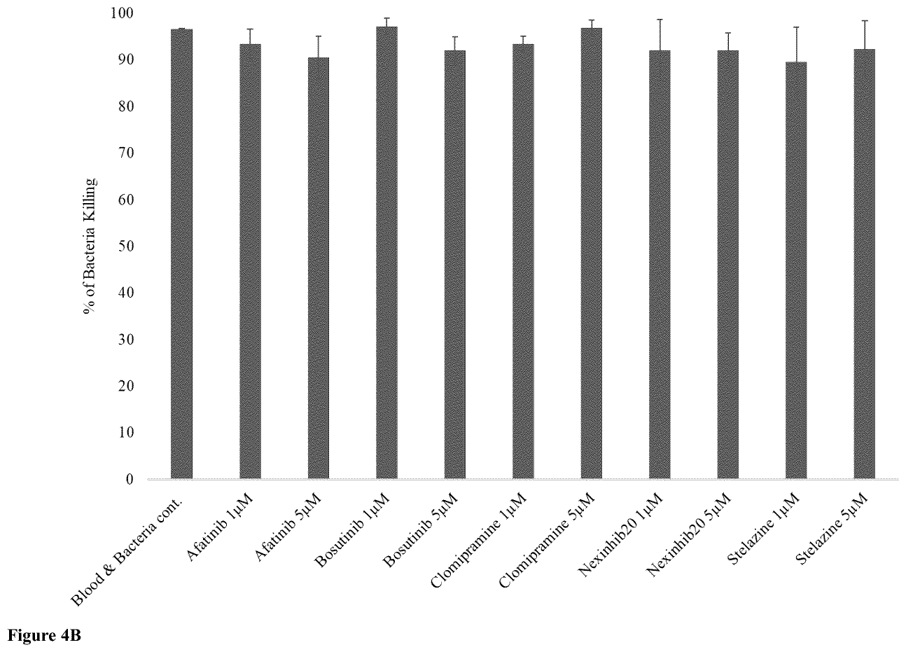

To test this hypothesis, neutrophils isolated from blood were cultured with the various drugs (concentration 1 uM) for 2 hours and then neutrophil survival was monitored by FACS. Cells that were double positive for Anexin V staining and PI staining were considered dead. PMA treatment alone resulted in nearly a 50% increase in the number of dead cells as compared to control, whereas none of the tested compounds produced more than a 6% increase in cell death (FIG. 4A). The known elastase inhibitor, Sivelestat, was used as a negative control and also showed less than a 6% increase. Thus, it was found that the drugs are not toxic to neutrophils and that the reduction in ROS is not due to increased neutrophil death.

Further, in order to test that ROS reduction did not impair neutrophil survival and function neutrophil bacteria killing was tested in the presence of the various molecules. E. Coli Bacteria was cultured in the presence of human blood together with the various compounds at a high (5 um) and low (1 uM) concentration at 37° C. for 4 hours, after which 10 uL was plated on LB plates and left overnight at 37° C. The percent of bacteria killed was counted on the plates as compared to bacteria grown without human blood. None of the tested drugs significantly reduced the blood's bacteria killing at either tested concentration (FIG. 4B). This indicates not only that the neutrophils were healthy and not killed/damaged by the drug, but also that the loss of ROS (though beneficial for surrounding tissue) did not impair the neutrophil's ability to carry out its main function of killing foreign cells. Taken together these results show that the various compounds are able to reduce toxic ROS and thus spare surrounding healthy tissue, while at the same time not killing neutrophils or inhibiting their ability to deal with infection/deleterious cells.

### Example 3: In Vivo Treatment of Lung Inflammation

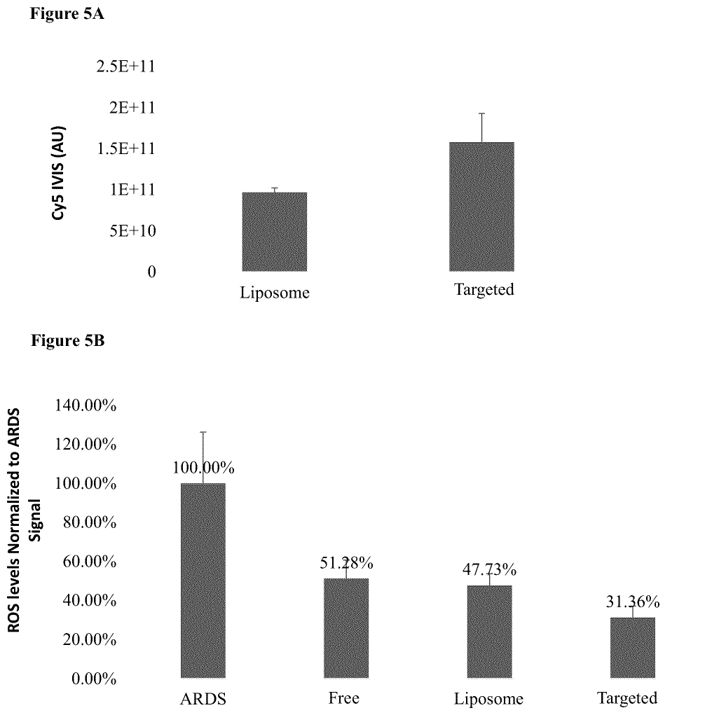

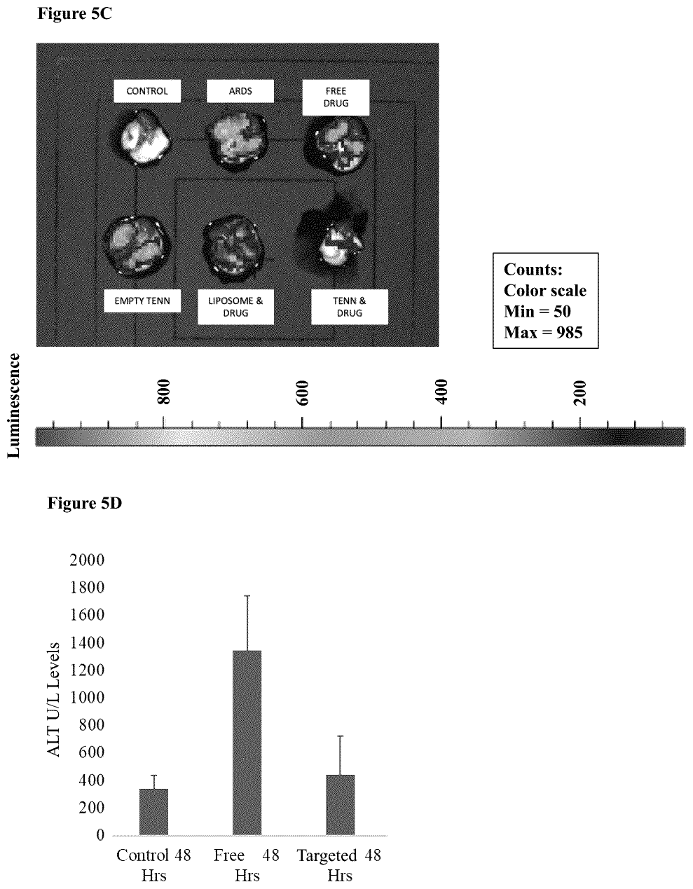

As Stelazine produced the strongest effect in whole blood treated with PMA, its ability to treat lung inflammation in an in vivo model was tested. Mice were given 150 ug LPS by inhalation and left for 2 days. Inhaled LPS is a well-known model for lung inflammation in general and can be used to model Acute respiratory distress syndrome (ARDS), Chronic obstructive pulmonary disease (COPD), acute lung injury (ALI) and emphysema among other lung conditions. The LPS was inhaled on Day 0, and then the dosing of the drugs was performed in the evening of day 1 and the morning of day 2. Three different formulations of Stelazine were tested by IV administration: free Stelazine administered in PBS, Stelazine loaded into liposomes in PBS, and Stelazine loaded into liposomes decorated with neutrophil targeting peptides in PBS. The peptide decorated liposomes were shown to localize to the inflamed lung better than naked liposomes (FIG. 5A) as indicated by Cy5 signal. When the effect on ROS production in the lungs was measured 48 hours after LPS administration, the free Stelazine and liposomal formulations were found to have strong effects producing about a 50% reduction in ROS as compared to the untreated control (FIG. 5B). The targeted liposomes produced a greater effect, with a nearly 70% reduction in ROS levels.

ARDS is known to induce lung dysfunction which manifests in the form of elevated ROS from neutrophils in the lungs. ROS was absent from healthy lungs but was strongly present in LPS treated lungs (FIG. 5C). Free Stelazine had an effect on ROS levels, which was also observed when the drug was loaded into naked liposomes. However, a much stronger effect was induced by Stelazine loaded into peptide decorated liposomes (FIG. 5C). Importantly, empty liposomes decorated with peptide had no effect on ALT levels. This data taken together demonstrates that Stelazine is a potent treatment of lung inflammation caused by neutrophil ROS, whose therapeutic efficacy is greatly enhanced by the use of neutrophil targeting with Stelazine.

One of the benefits of targeting of these drugs directly to neutrophils is to decrease their unwanted side effects. As mentioned early, many of these compounds have known side effects with respect to neutrophil differentiation in the bone marrow. Targeting to mature neutrophils decreases this unwanted side effect. To test if side effects are indeed decreased liver enzyme levels were measured in the mice after administration of free Stelazine and neutrophil targeted Stelazine. Free Stelazine increased alanine aminotransferase (ALT) levels in the blood indicated that liver damage was occurring (FIG. 5D) In contrast, the targeted liposomal Stelazine did not produce an increase in circulating levels of the enzyme. It is thus clear that neutrophil targeting is particularly beneficial for the selected compounds.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. (canceled)

2. (canceled)

3. A method of treating neutrophil-mediated inflammation or inhibiting neutrophil production of reactive oxygen species (ROS) in a subject in need thereof, the method comprising administering to said subject an agent selected from: a CDK4 inhibitor, a CDK6 inhibitor, an EGFR inhibitor, a SRC inhibitor and a Na channel blocker; thereby treating neutrophil-mediated inflammation in a subject.

4. The method of claim 3, wherein at least one of:
a. said neutrophil-mediated inflammation comprises neutrophil-mediated tissue damage;
b. said inflammation is mediated by ROS production by neutrophils;
c. said inflammation comprises elevated ROS from neutrophils; and
d. said inhibiting is inhibiting ROS production by activated neutrophils.

5. (canceled)

6. (canceled)

7. The method of claim 3, wherein said subject suffers from a disease or condition selected from an inflammatory disease, an autoimmune disease, cancer, acute tissue damage and an infectious disease, optionally wherein said disease or condition is selected from acute respiratory distress syndrome (ARDS), chronic obstructive pulmonary disease (COPD), acute lung injury (ALI), inflammatory bowel disease (IBD), sepsis, bacterial pneumonia, and viral pneumonia or said subject suffers from lung inflammation.

8. (canceled)

9. (canceled)

10. (canceled)

11. The method of claim 3, wherein said agent is a CDK4/6 dual inhibitor, optionally wherein said CDK4/6 dual inhibitor is selected from Abemaciclib, Ribociclib and Palbociclib.

12. (canceled)

13. The method of claim 3, wherein said agent is an EGFR inhibitor, optionally wherein said EGFR inhibitor is selected from Afatinib and Trifluoperazine.

14. (canceled)

15. The method of claim 3, wherein said agent is a SRC inhibitor, optionally wherein said SRC inhibitor is Bosutinib.

16. (canceled)

17. The method of claim 3, wherein said agent is a Na channel blocker, optionally wherein said Na channel blocker is selected from Dyclonine, Trifluoperazine, Clomipramine and Amitriptyline.

18. (canceled)

19. (canceled)

20. The method of claim 3, further comprising inhibiting elastase secretion from said neutrophils, optionally by administering an elastase secretion inhibitor.

21. The method of claim 3, wherein said agent is linked to or encapsulated by a nanoparticle, optionally wherein said nanoparticle comprises a neutrophil targeting moiety or wherein said nanoparticle comprises a peptide selected from SEQ ID NO: 1-13.

22. (canceled)

23. (canceled)

24. The method of claim 21, wherein said neutrophils are human neutrophils and said nanoparticle comprises a neutrophil targeting moiety selected from a peptide i-s-selected from SEQ ID NO: 1-8.

25. (canceled)

26. (canceled)

27. (canceled)

28. (canceled)

29. (canceled)

30. A method for treating neutrophil-mediated inflammation in a subject in need thereof, the method comprising administering to said subject a compound represented by Formula 1: 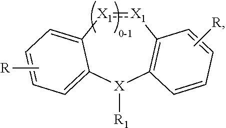
including any salt thereof, wherein:
each R is independently H, or represents one or more substituents independently comprising any one of —NO₂, —CN, —OR′, —CONH₂, —CONR′₂, —CNNR′₂, —CSNR′₂, —CONH—OR′, —CONH—NR′₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NR′NR′₂, —NR′NR′—OR′, —NNR′, carbonyl, C₁-C₁₀ haloalkyl, optionally substituted C₁-C₁₀ alkyl, —NH₂, —NR′₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted, as allowed by valency;
each X and X1 is independently selected from C, CR1, CH, CH2, N, NR1, NH, O, and S;
each R1 is independently H, or 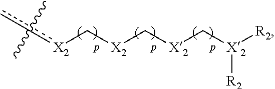
wherein each X₂ and X′₂ is
independently selected from CR, CH, CH₂, N, NR, NH, O, and S, as allowed by valency, or is absent;
optionally wherein any one of (i) two X2, (ii) two X′2 are interconnected by a covalent bond, forming a carbocyclic ring, optionally comprising one or more heteroatoms;
p is between 0 and 10;
each R2 is independently H, absent, or is selected from the group comprising an optionally substituted C1-C10 alkyl, an C1-C10 alkyl-aryl, an C1-C10 alkyl-cycloalkyl, optionally substituted C3-C10 cycloalkyl, optionally substituted C3-C10 heterocyclyl, optionally substituted heteroaryl, optionally substituted aryl, —NO2, —CN, —OR′, —CONH2, —CONR′2, —CNNR′2, —CSNR′2, —CONH—OR′, —CONH—NR′2, —NHCOR′, —NHCSR′, —NHCNR′, —NC(═O)R′, —NC(═O)OR′, —NC(═O)NR′, —NC(═S)OR′, —NC(═S)NR′, —SO2R′, —SOR′, —SR′, —SO2OR′, —SO2N(R′)2, —NR′NR′2, —NR′NR′—OR′, —NNR′, carbonyl, C1-C10 haloalkyl, optionally substituted C1-C10 alkyl, —NH2, —NR′2, —NH(C1-C10 alkyl), —N(C1-C10 alkyl)2, C1-C10 haloalkoxy, hydroxy(C1-C10 alkyl), hydroxy(C1-C10 alkoxy), alkoxy(C1-C10 alkyl), alkoxy(C1-C10 alkoxy), amino(C1-C10 alkyl), —CONH(C1-C10 alkyl), —CON(C1-C10 alkyl)2, —CO2H, —CO2R′, —OCOR′, —C(═O)R′, —OC(═O)OR′, —OC(═O)NR′, —OC(═S)OR′, —OC(═S)NR′, a heteroatom, (C1-C10 alkyl)alkyl-cycloalkyl, (C1-C10 alkyl)alkyl-aryl, (C1-C10 alkyl)alkyl-heteroaryl, or a combination thereof as allowed by valency;
or wherein both R2 are interconnected by a covalent bond, forming a carbocyclic ring, optionally comprising one or more heteroatoms;
each R′ independently represents hydrogen, is absent, or is selected from the group comprising optionally substituted C1-C10 alkyl, an C1-C10 alkyl-aryl, —NH2, —OR′, —OH, —CONH2, —CONR′2, —CNNR′2, —CSNR′2, —CONH—OR′, —CONH—NR′2, —NHCOR′, —NHCSR′, —NHCNR′, —NC(═O)R′, —NC(═O)OR′, —NC(═O)NR′, —NC(═S)OR′, —NC(═S)NR′, —SO2R′, —SOR′, —SR′, —SO2OR′, —SO2N(R′)2, —NR′NR′2, —NR′NR′—OR′, —NNR′, carbonyl, an C1-C10 alkyl-cycloalkyl, optionally substituted C3-C10 cycloalkyl, optionally substituted C3-C10 heterocyclyl, optionally substituted heteroaryl, optionally substituted aryl, or a combination thereof as allowed by valency; and wherein at least one R1 comprises an amine.

31. (canceled)

32. The method of claim 30, wherein at least one of:
a. said compound is characterized by a pKa between about 8 and about 10;
b. said neutrophil-mediated inflammation comprises neutrophil-mediated tissue damage;
c. said inflammation, is mediated by ROS production by neutrophils;
d. said inflammation comprises elevated liver enzyme levels, optionally wherein said liver enzyme is alanine aminotransferase (ALT).

33. The method of claim 30, wherein at least one R1 or comprises said substituent or is 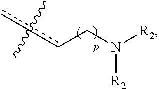
wherein p is between 1 and 5; and wherein each R2 is independently H, absent, or is selected from the group comprising an optionally substituted C1-C10 alkyl, an C1-C10 alkyl-aryl, an C1-C10 alkyl-cycloalkyl, optionally substituted C3-C10 cycloalkyl, optionally substituted C3-C10 heterocyclyl, optionally substituted heteroaryl, optionally substituted aryl, OR′, CONH2, CONR′2, CNNR′2, CSNR′2, CONH—OR′, CONH—NR′2, NHCOR′, NHCSR′, NHCNR′, —NC(═O)R′, —NC(═O)OR′, —NC(═O)NR′, —NC(═S)OR′, —NC(═S)NR′, SO2R′, SOR′, —SR′, SO2OR′, SO2N(R′)2, —NR′NR′2, —NR′NR′—OR′, —NNR′, carbonyl, C1-C10 haloalkyl, optionally substituted C1-C10 alkyl, NH2, NR′2, NH(C1-C10 alkyl), N(C1-C10 alkyl)2, C1-C10 haloalkoxy, hydroxy(C1-C10 alkyl), hydroxy(C1-C10 alkoxy), alkoxy(C1-C10 alkyl), alkoxy(C1-C10 alkoxy), amino(C1-C10 alkyl), CONH(C1-C10 alkyl), CON(C1-C10 alkyl)2, CO2H, CO2R′, —OCOR′, —C(═O)R′, —OC(═O)OR′, —OC(═O)NR′, —OC(═S)OR′, —OC(═S)NR′, a heteroatom, (C1-C10 alkyl)alkyl-cycloalkyl, (C1-C10 alkyl)alkyl-aryl, (C1-C10 alkyl)alkyl-heteroaryl, or a combination thereof as allowed by valency;
or wherein both R2 are interconnected by a covalent bond, forming a 5-6 membered ring comprising one or more heteroatoms.

34. The method of claim 33, wherein said compound is represented by Formula 2: 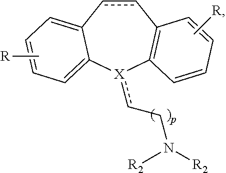
or by Formula 2A: 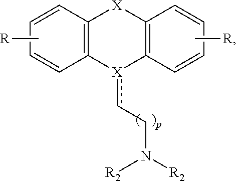
wherein each X is independently selected from C, CH, CH2, N, NR1, NH, O, and S.

35. The method of claim 30, wherein said compound is or comprises any one of: 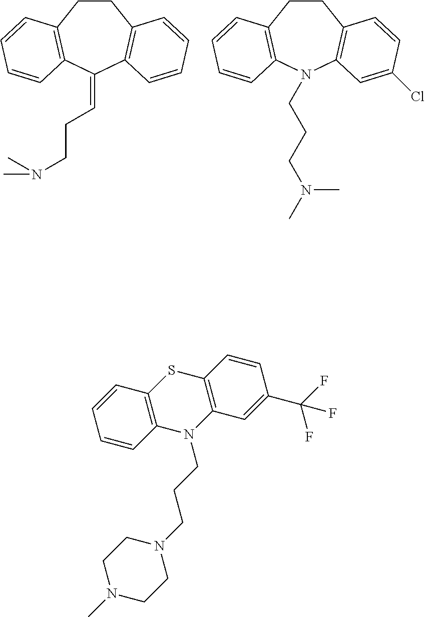
including any salt, or any combination thereof.

36. (canceled)

37. (canceled)

38. (canceled)

39. The method of claim 30, wherein said subject suffers from a disease selected from an inflammatory disease, an autoimmune disease, cancer and an infectious disease, optionally wherein said disease is selected from acute respiratory distress syndrome (ARDS), chronic obstructive pulmonary disease (COPD), inflammatory bowel disease (IBD), sepsis, bacterial pneumonia, and viral pneumonia or said subject suffers from lung inflammation.

40. (canceled)

41. (canceled)

42. (canceled)

43. A pharmaceutical composition comprising a nanoparticle comprising an agent and a neutrophil targeting peptide; wherein said agent is selected from a cyclin dependent kinase 4 (CDK4) inhibitor, a cyclin dependent kinase 6 (CDK6) inhibitor, an epidermal growth factor receptor (EGFR) inhibitor, a proto-oncogene tyrosine-protein kinase Src (SRC) inhibitor and a sodium (Na) channel blocker or is a compound represented by Formula 1: 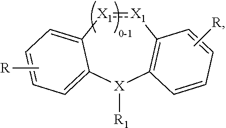
including any salt thereof, wherein:
each R is independently H, or represents one or more substituents independently comprising any one of —NO₂, —CN, —OR′, —CONH₂, —CONR′₂, —CNNR′₂, —CSNR′₂, —CONH—OR′, —CONH—NR′₂, —NHCOR′, —NHCSR′, —NHCNR′, —NC(═O)R′, —NC(═O)OR′, —NC(═O)NR′, —NC(═S)OR′, —NC(═S)NR′, —SO₂R′, —SOR′, —SR′, —SO₂OR′, —SO₂N(R′)₂, —NR′NR′₂, —NR′NR′—OR′, —NNR′, carbonyl, C₁-C₁₀ haloalkyl, optionally substituted C₁-C₁₀ alkyl, —NH₂, —NR′₂, —NH(C₁-C₁₀ alkyl), —N(C₁-C₁₀ alkyl)₂, C₁-C₁₀ haloalkoxy, hydroxy(C₁-C₁₀ alkyl), hydroxy(C₁-C₁₀ alkoxy), alkoxy(C₁-C₁₀ alkyl), alkoxy(C₁-C₁₀ alkoxy), amino(C₁-C₁₀ alkyl), —CONH(C₁-C₁₀ alkyl), —CON(C₁-C₁₀ alkyl)₂, —CO₂H, —CO₂R′, —OCOR′, —C(═O)R′, —OC(═O)OR′, —OC(═O)NR′, —OC(═S)OR′, —OC(═S)NR′, a heteroatom, cycloalkyl, heterocyclyl aryl, heteroaryl, (C₁-C₁₀ alkyl)alkyl-cycloalkyl, (C₁-C₁₀ alkyl)alkyl-aryl, (C₁-C₁₀ alkyl)alkyl-heteroaryl, or any combination thereof, and wherein each of cycloalkyl, heterocyclyl aryl, heteroaryl is substituted or non-substituted, as allowed by valency;
each X and X1 is independently selected from C, CR1, CH, CH2, N, NR1, NH, O, and S;
each R1 is independently H, or 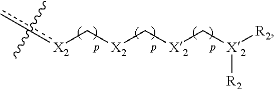
wherein each X₂ and X′₂ is independently selected from CR, CH, CH₂, N, NR, NH, O, and S, as allowed by valency, or is absent;
optionally wherein any one of (i) two X2, (ii) two X′2 are interconnected by a covalent bond, forming a carbocyclic ring, optionally comprising one or more heteroatoms;
p is between 0 and 10;
each R2 is independently H, absent, or is selected from the group comprising an optionally substituted C1-C10 alkyl, an C1-C10 alkyl-aryl, an C1-C10 alkyl-cycloalkyl, optionally substituted C3-C10 cycloalkyl, optionally substituted C3-C10 heterocyclyl, optionally substituted heteroaryl, optionally substituted aryl, —NO2, —CN, —OR′, —CONH2, —CONR′2, —CNNR′2, —CSNR′2, —CONH—OR′, —CONH—NR′2, —NHCOR′, —NHCSR′, —NHCNR′, —NC(═O)R′, —NC(═O)OR′, —NC(═O)NR′, —NC(═S)OR′, —NC(═S)NR′, —SO2R′, —SOR′, —SR′, —SO2OR′, —SO2N(R′)2, —NR′NR′2, —NR′NR′—OR′, —NNR′, carbonyl, C1-C10 haloalkyl, optionally substituted C1-C10 alkyl, —NH2, —NR′2, —NH(C1-C10 alkyl), —N(C1-C10 alkyl)2, C1-C10 haloalkoxy, hydroxy(C1-C10 alkyl), hydroxy(C1-C10 alkoxy), alkoxy(C1-C10 alkyl), alkoxy(C1-C10 alkoxy), amino(C1-C10 alkyl), —CONH(C1-C10 alkyl), —CON(C1-C10 alkyl)2, —CO2H, —CO2R′, —OCOR′, —C(═O)R′, —OC(═O)OR′, —OC(═O)NR′, —OC(═S)OR′, —OC(═S)NR′, a heteroatom, (C1-C10 alkyl)alkyl-cycloalkyl, (C1-C10 alkyl)alkyl-aryl, (C1-C10 alkyl)alkyl-heteroaryl, or a combination thereof as allowed by valency;
or wherein both R2 are interconnected by a covalent bond, forming a carbocyclic ring, optionally comprising one or more heteroatoms;
each R′ independently represents hydrogen, is absent, or is selected from the group comprising optionally substituted C1-C10 alkyl, an C1-C10 alkyl-aryl, —NH2, —OR′, —OH, —CONH2, —CONR′2, —CNNR′2, —CSNR′2, —CONH—OR′, —CONH—NR′2, —NHCOR′, —NHCSR′, —NHCNR′, —NC(═O)R′, —NC(═O)OR′, —NC(═O)NR′, —NC(═S)OR′, —NC(═S)NR′, —SO2R′, —SOR′, —SR′, —SO2OR′, —SO2N(R′)2, —NR′NR′2, —NR′NR′—OR′, —NNR′, carbonyl, an C1-C10 alkyl-cycloalkyl, optionally substituted C3-C10 cycloalkyl, optionally substituted C3-C10 heterocyclyl, optionally substituted heteroaryl, optionally substituted aryl, or a combination thereof as allowed by valency; and wherein at least one R1 comprises an amine, and wherein said neutrophil targeting peptide comprises a sequence selected from SEQ ID NO: 1-13; optionally wherein at least one R1 is or comprises said substituent.

44. (canceled)

45. (canceled)

46. The pharmaceutical composition of claim 43, wherein at least one of:
a. said compound is characterized by a pKa between about 8 and about 10;
b. at least one R1 is 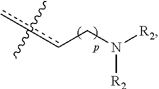
wherein p is between 1 and 5; and wherein each R2 is independently H, absent, or is selected from the group comprising an optionally substituted C1-C10 alkyl, an C1-C10 alkyl-aryl, an C1-C10 alkyl-cycloalkyl, optionally substituted C3-C10 cycloalkyl, optionally substituted C3-C10 heterocyclyl, optionally substituted heteroaryl, optionally substituted aryl, OR′, CONH2, CONR′2, CNNR′2, CSNR′2, CONH—OR′, CONH—NR′2, NHCOR′, NHCSR′, NHCNR′, —NC(═O)R′, —NC(═O)OR′, —NC(═O)NR′, —NC(═S)OR′, —NC(═S)NR′, SO2R′, SOR′, —SR′, SO2OR′, SO2N(R′)2, —NR′NR′2, —NR′NR′—OR′, —NNR′, carbonyl, C1-C10 haloalkyl, optionally substituted C1-C10 alkyl, NH2, NR′2, NH(C1-C10 alkyl), N(C1-C10 alkyl)2, C1-C10 haloalkoxy, hydroxy(C1-C10 alkyl), hydroxy(C1-C10 alkoxy), alkoxy(C1-C10 alkyl), alkoxy(C1-C10 alkoxy), amino(C1-C10 alkyl), CONH(C1-C10 alkyl), CON(C1-C10 alkyl)2, CO2H, CO2R′, —OCOR′, —C(═O)R′, —OC(═O)OR′, —OC(═O)NR′, —OC(═S)OR′, —OC(═S)NR′, a heteroatom, (C1-C10 alkyl)alkyl-cycloalkyl, (C1-C10 alkyl)alkyl-aryl, (C1-C10 alkyl)alkyl-heteroaryl, or a combination thereof as allowed by valency;
or wherein both R2 are interconnected by a covalent bond, forming a 5-6 membered ring comprising one or more heteroatoms.

47. The pharmaceutical composition of claim 46, wherein said compound is represented by Formula 2: 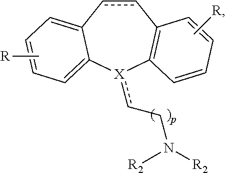
or by Formula 2A: 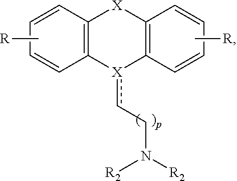
wherein each X is independently selected from C, CH, CH2, N, NR1, NH, O, and S.

48. The pharmaceutical composition of claim 43, wherein said compound is or comprises any one of: 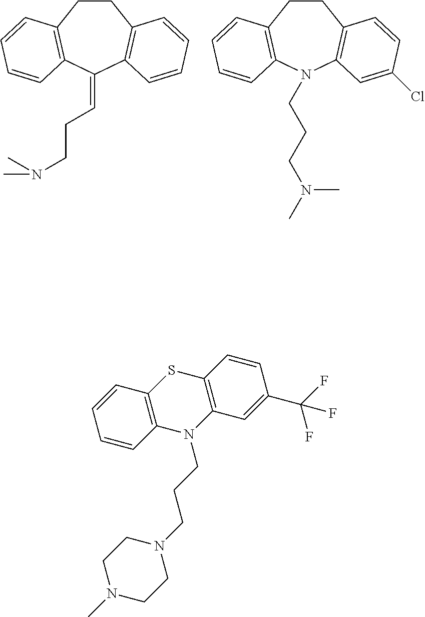
including any salt, or any combination thereof.

49. (canceled)


SEQUENCE LISTING (16 sequences)

SEQ ID NO: 1 - protein, 15 aa, synthetic construct
KFPDLDSRRLPHMSL

SEQ ID NO: 2 - protein, 12 aa, synthetic construct
LATTHMVFSPDH

SEQ ID NO: 3 - protein, 13 aa, synthetic construct
PSSNLESTPLSLL

SEQ ID NO: 4 - protein, 13 aa, synthetic construct
SSLMTTQLIATSI

SEQ ID NO: 5 - protein, 12 aa, synthetic construct
PELDSKPYFPPL

SEQ ID NO: 6 - protein, 12 aa, synthetic construct
ELVTASMPRPNN

SEQ ID NO: 7 - protein, 12 aa, synthetic construct
SLESSPMAQLPQ

SEQ ID NO: 8 - protein, 12 aa, synthetic construct
SELRSTPLLVPS

SEQ ID NO: 9 - protein, 10 aa, synthetic construct
LQIQSWSSSP

SEQ ID NO: 10 - protein, 10 aa, synthetic construct
STMTILGTGS

SEQ ID NO: 11 - protein, 12 aa, synthetic construct
TETSLRIVSTNP

SEQ ID NO: 12 - protein, 12 aa, synthetic construct
LSIVSGSALNHL

SEQ ID NO: 13 - protein, 10 aa, synthetic construct
LTLVSERPMI

SEQ ID NO: 14 - protein, 4 aa, synthetic construct
GGGS

SEQ ID NO: 15 - protein, 5 aa, synthetic construct
GGGGS

SEQ ID NO: 16 - protein, 4 aa, synthetic construct
KAKA